SEQUENCE LISTING (1 sequence)

SEQ ID NO: 1 - protein, 20 aa, Artificial Sequence
  Synthetic Peptide
ARTKQTARKSTGGKAPRKQL

(19) United States
(12) Patent Application Publication
(10) Pub. No.: US 2019/0322643 A1
(43) Pub. Date: Oct. 24, 2019

(54) HISTONE DEACETYLASE AND HISTONE METHYLTRANSFERASE INHIBITORS AND METHODS OF MAKING AND USE OF THE SAME

(71) Applicant: Georgia State University Research Foundation, Inc., Atlanta, GA (US)

(72) Inventors: Peng George Wang, Alpharetta, GA (US); Muhammed Shukkoor Kondengaden, Atlanta, GA (US); Qing Zhang, Atlanta, GA (US); Lanlan Zang, Decatur, GA (US)

(21) Appl. No.: 16/314,535

(22) PCT Filed: Jun. 29, 2017
(86) PCT No.: PCT/US2017/040007

§ 371 Date: Dec. 31, 2018

Related U.S. Application Data
(60) Provisional application No. 62/356,124, filed on Jun. 29, 2016.

(51) Int. Cl. C07D 401/12; C07D 401/14; C07D 403/04; A61P 35/00

(52) U.S. Cl. CPC C07D 401/12; A61P 35/00; C07D 403/04; C07D 401/14

(57) ABSTRACT

Inhibitors of HDAC and G9a inhibitors such as, for example, single compounds that inhibitor both HDAC and methyltransferase G9a (referred to herein as dual HDAC-G9a inhibitors, dual HDAC-G9a compounds, and dual HDAC-G9a inhibitor compounds) are described herein. For example, dual inhibitor compounds of Formulae I-III and methods of making and using thereof, are described herein. The dual inhibition activity of these compounds can provide inhibition of both histone deacetylase (HDAC) and histone methyltransferase G9a thereby providing anti-cancer effects.

### CROSS-REFERENCE TO RELATED APPLICATIONS

This application claims the benefit of and priority to U.S. Ser. No. 62/356,124 filed Jun. 29, 2016 and which is incorporated by reference in its entirety.

### REFERENCE TO SEQUENCE LISTING

The Sequence Listing submitted as a text file named “GSURF_2016_10_PCT_ST25.txt,” created on Jun. 28, 2017, and having a size of 735 bytes is hereby incorporated by reference pursuant to 37 C.F.R § 1.52(e)(5).

### FIELD OF THE INVENTION

The present invention generally relates to inhibitors of histone deacetylase (HDAC) and histone methyltransferase G9a such as, for example, single compounds that inhibitor both HDAC and methyltransferase G9a, and methods of making and using thereof.

### BACKGROUND OF THE INVENTION

Cancer is a disease with difficult treatment options due to the multifactorial basis of initiation and progression. A treatment targeting multiple components instead of a single component would therefore be of particular interest in cancer therapeutics. Two classes of small molecules which target the enzymes histone deacetylases (HDACs) and histone methyltransferase G9a, which are both key posttranslational enzymes in cancer development as described below.

Histone deacetylases (HDACs) fall into the category of eraser enzymes, so termed due to their ability to reverse the acetylation modification employed by another enzyme histone acetyl transferases (HATs) (Batty et al., Cancer Lett. 2009, 280, 192-200). Aberrant activity of HDACs has been well documented in several cancer phenotypes, with HDAC inhibitors (HDACIs) shown to be antineoplastic agents. HDACIs have multiple cell type-specific effects in vitro and in vivo, such as growth arrest, cell differentiation, and apoptosis in malignant cells (Dokmanovic et al., Mol Cancer Res. 2007, 5, 981-989; Botrugno et al., Cancer Lett. 2009, 280, 134-44). HDACIs have been shown to induce apoptosis in both solid and hematological malignancies using both transcription dependent and transcription independent mechanisms (Duan et al., Mol. Cell. Biol. 2005, 25, 1608-1619; Lai et al., J. Med. Chem. 2012, 55, 3777-91; Luchenko et al., Mol. Oncol. 2014).

Of interest is the PKMT G9a (also known as KMT1C, EHMT2), which is a histone 3 lysine 9 (H3K9) specific methyltransferase that is overexpressed in many cancers including leukemia, hepatocellular carcinoma, and lung cancer. G9a is notable for its role in cancer cell proliferation and knockdown of G9a in prostate, lung and leukemia cancer cells resulted in the inhibition of cell growth (Liu et al., J. Med. Chem. 2013, 56, 8931-8942; Vedadi et al., Nat. Chem. Biol. 2011, 7, 566-574; Spannhoff et al., ChemMedChem. 2009, 4, 1568-1582). Presently, there are a number of small molecules with varying structural cores that have been found to inhibit G9a which are also under consideration in clinical trials (Liu et al., J. Med. Chem. 2013, 56, 8931-8942; Sweis et al., ACS Med Chem. Lett. 2014, 5, 205-209).

Accordingly, there is a need for new classes of small molecules that can target the enzymes histone deacetylases (HDACs) and histone methyltransferase G9a, both of which are key posttranslational enzymes in cancer development.

It is therefore an object of the invention to provide new HDAC and G9a inhibitors such as, for example, single compounds that inhibitor both HDAC and methyltransferase G9a.

It is a further object of the invention to provide new anti-cancer agents such as HDAC and G9a inhibitors such as, for example, single compounds that inhibitor both HDAC and methyltransferase G9a.

It is a further object of the invention to provide methods of making and using HDAC and G9a inhibitors such as, for example, single compounds that inhibitor both HDAC and methyltransferase G9a.

It is a further object of the invention to provide methods of treating cancer with anti-cancer agents such as HDAC and G9a inhibitors such as, for example, single compounds that inhibitor both HDAC and methyltransferase G9a.

### SUMMARY OF THE INVENTION

Inhibitors of HDAC and G9a inhibitors such as, for example, single compounds that inhibitor both HDAC and methyltransferase G9a (referred to herein as dual HDAC-G9a inhibitors, dual HDAC-G9a compounds, and dual HDAC-G9a inhibitor compounds) are described herein. For example, dual HDAC-G9a inhibitor compounds according to Formulae I, II, or II, and methods of making and using thereof, are described herein.

In some forms, the dual inhibitor compounds are defined according to Formula I:

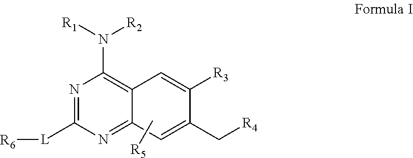

where X is absent or oxygen (O), nitrogen (NH or NR₁₈) or sulfur (S);

where R₁ is hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or one of the moieties:

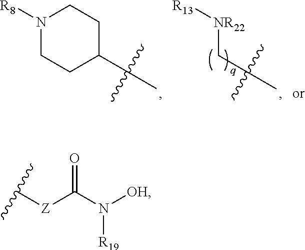

where q is an integer value in the range of 1-15, more preferably 1-10, most preferably 1-5;

where R₄, R₆, R₈, and R₁₃ are independently hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

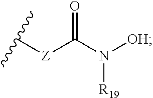

where Z is absent or a linking moiety, where the linking moiety is oxygen (O), nitrogen (NR₂₃), sulfur (S), optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where L is absent or a linking moiety, wherein the linking moiety is optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where R₂, R₃, R₅, R₁₈, R₁₉, R₂₂, and R₂₃ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and

where at least one of R₁, R₄, R₆, R₈, or R₁₃ is the moiety:

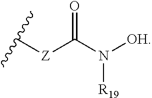

In certain forms of compounds according to Formula I, Z is:

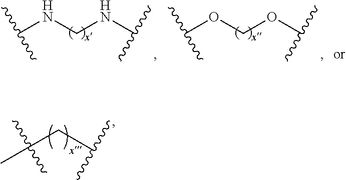

where x′, x″, and x′″ are integer values independently in the range of 1-15, more preferably 1-10, most preferably 1-5.

In certain other forms of compounds according to Formula I, Z is absent and R₆ is:

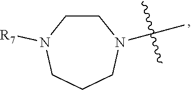

where R₇ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and the remaining groups/variables are as previously defined.

In other forms, the dual inhibitor compounds are defined according to Formula II:

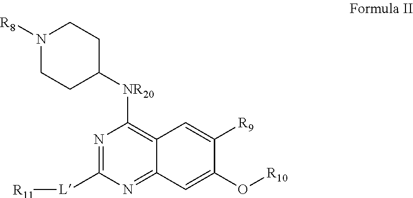

where R₈, R₁₀, and R₁₁ are independently hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

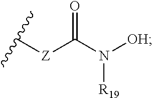

where Z is absent or a linking moiety, wherein the linking moiety is oxygen (O), nitrogen (NR₂₃), sulfur (S), optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where L′ is absent or a linking moiety, wherein the linking moiety is optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where R₉, R₂₀, R₂₃, and R₁₉ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and where at least one of R₈, R₁₀, or R₁₁ is the moiety:

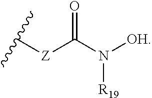

In some forms of compounds according to Formula II, R₈ is an optionally substituted benzyl.

In certain forms of compounds according to Formula II, Z is:

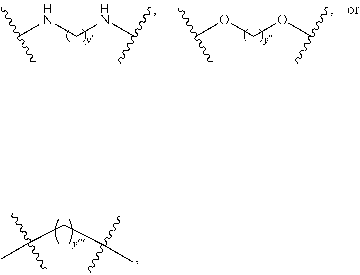

where y′, y″, and y′″ are independently an integer value in the range of 1-15, more preferably 1-10, most preferably 1-5.

In certain other forms of compounds according to Formula II, Z is

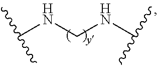

where y′ is as defined above;

R₁₁ is:

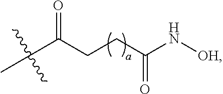

where a is an integer value in the range of 1-15, more preferably 1-10, most preferably 1-5; and

R₈, R₉, and R₁₀ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl. In preferred forms, R₈ and R₁₀ are an optionally substituted alkyl, optionally substituted aryl, or optionally substituted heteroaryl, and R₉ is hydrogen or an optionally substituted alkoxyl.

In yet other forms of compounds according to Formula II, Z is absent and R₁₁ is:

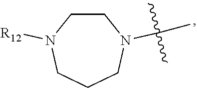

where R₁₂ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and the remaining groups/variables are as previously defined.

In certain forms of compounds according to Formula II, Z is absent;

R₁₁ is:

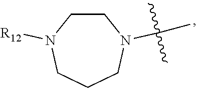

where R₁₂ is as previously defined; and

R₈ is:

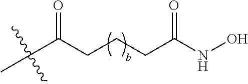

where b is an integer value in the range of 1-15, more preferably 1-10, most preferably 1-5; and

R₉ and R₁₀ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl.

In preferred forms, R₉ is an optionally substituted alkoxyl and R₁₀ is an optionally substituted alkyl, optionally substituted aryl, or optionally substituted heteroaryl.

In yet other forms of compounds according to Formula II, Z is absent;

R₁₁ is:

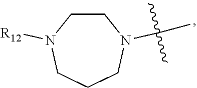

where R₁₂ is as previously defined; and

R₁₀ is:

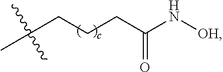

where c is an integer value in the range of 1-15, more preferably 1-10, most preferably 1-5; and

R₈ and R₉ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl. In preferred forms, R₈ is an optionally substituted alkyl, optionally substituted aryl, or optionally substituted heteroaryl, and R₉ is an optionally substituted alkoxyl.

In still other forms, the dual inhibitor compounds are defined according to Formula III:

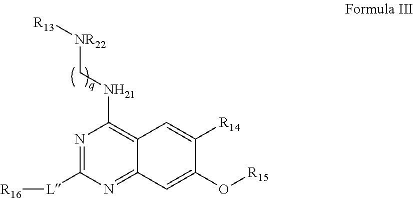

where q is an integer in the range of 1-15, more preferably 1-10, most preferably 1-5;

where R₁₃, R₁₅, and R₁₆ are independently hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

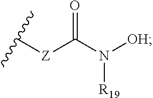

where Z is absent or a linking moiety, where the linking moiety is oxygen (O), nitrogen (NR₂₃), sulfur (S), optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where L″ is absent or a linking moiety, wherein the linking moiety is optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and

where R₁₄, R₂₁, R₂₂, R₂₃, and R₁₉ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and

at least one of R₁₃, R₁₅, or R₁₆ is the moiety:

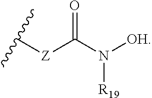

In certain forms of compounds according to Formula III, Z is:

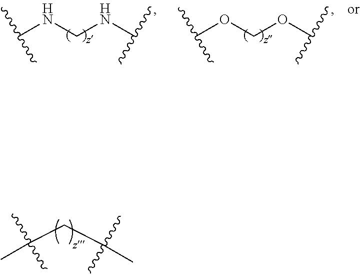

where z′, z″, and z′″ are an integer value in the range of 1-15, more preferably 1-10, most preferably 1-5.

In other forms of compounds according to Formula III, Z is absent and R₁₆ is:

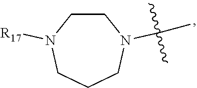

where R₁₇ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and the remaining groups/variables are as previously defined.

In certain forms of compounds according to Formula III, Z is absent;

R₁₆ is:

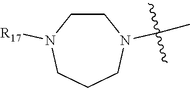

where R₁₇ is as previously defined; q is 2;

R₁₃ is:

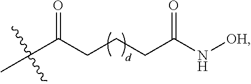

where d is an integer value in the range of 1-15, more preferably 1-10, most preferably 1-5; and

R₁₄ and R₁₅ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl. In preferred forms, R₁₄ a hydrogen or an optionally substituted alkoxyl and R₁₅ is an optionally substituted alkyl, optionally substituted aryl, or optionally substituted heteroaryl.

The dual inhibitor compounds described herein can be administered as, for example, the free acid or base, or as a pharmaceutically acceptable salt, prodrug, or solvate. The compounds can be used as, for example, anti-cancer agents in a method of treatment of a patient in need thereof to prevent, inhibit, or treat cancer. In some embodiments, the dual inhibitor compounds described herein can be used to treat diseases such as fungal infections, Alzheimer's disease, Huntington's disease, epilepsy, depression, inflammatory diseases, and HIV, all of which are affected by HDACs.

The dual inhibitor compounds described herein can be formulated with, for example, a pharmaceutically acceptable carrier and, optionally one or more pharmaceutically acceptable excipients, for administration to a patient in need thereof.

### BRIEF DESCRIPTION OF THE DRAWINGS

FIGS. 1A-1D are graphs showing the effect of compound 14 on biochemical and cell assays. FIG. 1A shows the methylation pattern observed via MALDI-TOF after incubating with inhibitor compound 14 and BIX-01294 for 30 minutes. FIG. 1B shows the percent (%) ratio of the H3K9Me0, H3K9Me1 and H3K9Me2 after incubating 30 minutes with compound 14 and BIX-01294 versus no inhibitor. FIG. 1C shows the In Cell Western (ICW) assay of compound 14 and BIX-01294 in MDA-MB 231 cell lines. FIG. 1D shows the result of homogenous histone deacetylase assay of compound 14 alongside SAHA in K562 cell lines.

### DETAILED DESCRIPTION OF THE INVENTION

### Definitions

The singular forms “a,” “an,” and “the” include plural reference unless the context clearly dictates otherwise. For example, reference to “a compound” includes a plurality of compounds and reference to “the compound” is a reference to one or more compounds and equivalents thereof known to those skilled in the art.

The term “effective amount” refers to any amount that results in a predetermined or desired outcome. For example, the pharmaceutical compositions or formulations described herein can contain an effective amount of a dual function HDAC-G9a inhibitor in order to treat a cancer to result in, for example, inhibition of the cancer or reduction in tumor size. Other outcomes may also occur in addition to and/or in combination with the ones listed.

As used herein, the term “analog” refers to a chemical compound with a structure similar to that of another (reference compound) but differing from it in respect to a particular component, functional group, atom, etc. As used herein, the term “derivative” refers to compounds which are formed from a parent compound by chemical reaction(s). These differences in suitable analogues and derivatives include, but are not limited to, replacement of one or more functional groups on the ring with one or more different functional groups or reacting one or more functional groups on the ring to introduce one or more substituents.

Numerical ranges disclosed in the present application of any type, disclose individually each possible number that such a range could reasonably encompass, as well as any sub-ranges and combinations of sub-ranges encompassed therein. A carbon range (i.e., C₁-C₁₀), is intended to disclose individually every possible carbon value and/or sub-range encompassed within. For example, a carbon length range of C₁-C₁₀ discloses C₁, C₂, C₃, C₄, C₅, C₆, C₇, C₈, C₉, and C₁₀, as well as discloses sub-ranges encompassed therein, such as C₂-C₉, C₃-C₈, C₁-C₅, etc.

“Aryl”, as used herein, refers to 5-, 6- and 7-membered aromatic, heterocyclic, fused aromatic, fused heterocyclic, biaromatic, or biheterocyclic ring system, optionally substituted by halogens, alkyl-, alkenyl-, and alkynyl-groups. Broadly defined, “Ar”, as used herein, includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “Ar” also includes polycyclic ring systems having two or more cyclic rings in which two or more carbons are common to two adjoining rings (the rings are “fused rings”) where at least one of the rings is aromatic, e.g., the other cyclic rings can be cycloalkyls, cycloalkenyls, cycloalkynyls, aryls and/or heterocycles.

Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

“Alkylaryl”, as used herein, refers to an alkyl group substituted with an aryl group (e.g., an aromatic or heteroaromatic group).

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) where Y is absent or is H, O, (C₁₋₄)alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Heteroaryl”, as used herein, refers to a monocyclic aromatic ring containing five or six ring atoms consisting of carbon and 1, 2, 3, or 4 heteroatoms each selected from the group consisting of non-peroxide oxygen, sulfur, and N(Y) where Y is absent or is H, O, (C₁-C₈)alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl can be furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

“Halogen”, as used herein, refers to fluorine, chlorine, bromine, or iodine.

The terms “alkenyl” and “alkynyl” refer to unsaturated aliphatic groups analogous in length and possible substitution to the alkyls described above, but that contain at least one double or triple bond respectively.

The terms ortho, meta and para apply to 1,2-, 1,3- and 1,4-disubstituted benzenes, respectively. For example, the names 1,2-dimethylbenzene and ortho-dimethylbenzene are synonymous.

“Substituted”, as used herein, means that the functional group contains one or more substituents attached thereon including, but not limited to, hydrogen, halogen, cyano, alkoxyl, alkyl, alkenyl, cycloalkyl, cycloalkenyl, aryl, heterocycloalkyl, heteroaryl, amine, hydroxyl, oxo, formyl, acyl, carboxylic acid (—COOH), —C(O)R′, —C(O)OR′, carboxylate (—COO—), primary amide (e.g., —CONH₂), secondary amide (e.g., —CONHR′), —C(O)NR′R″, —NR′R″, —NR'S(O)₂R″, —NR′C(O)R″, —S(O)₂R″, —SR′, and —S(O)₂NR′R″, sulfinyl group (e.g., —SOR′), and sulfonyl group (e.g., —SOOR′); where R′ and R″ may each independently be hydrogen, alkyl, alkenyl, alkynyl, cycloalkyl, aryl, heterocycloalkyl and heteroaryl; where each of R′ and R″ is optionally independently substituted with one or more substituents selected from the group consisting of halogen, hydroxyl, oxo, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or oxo or alkyl optionally substituted with hydroxyl, cycloalkyl optionally substituted with hydroxyl, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl, or combinations thereof. In some instances, “substituted” also refers to one or more substitutions of one or more of the carbon atoms in a carbon chain (i.e., alkyl, alkenyl, cycloalkyl, cycloalkenyl, and aryl groups) which can be substituted by a heteroatom, such as, but not limited to, a nitrogen or oxygen.

“Pharmaceutically acceptable salt”, as used herein, refer to derivatives of the compounds described herein where the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Solvate”, as used herein, refers to a compound which is formed by the interaction of molecules of a solute with molecules of a solvent.

As used herein, “inhibit” or other forms of the word such as “inhibiting” or “inhibition” means to hinder or restrain a particular characteristic. It is understood that this is typically in relation to some standard or expected value, in other words it is relative, but that it is not always necessary for the standard or relative value to be referred to.

As used herein, “treatment” or “treating” means to administer a composition to a subject or a system with an undesired condition. The condition can include a disease. “Prevention” or “preventing” means to administer a composition to a subject or a system at risk for the condition. The condition can include a predisposition to a disease. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur. It is understood that where treat or prevent are used, unless specifically indicated otherwise, the use of the other word is also expressly disclosed.

As used herein, “subject,” “individual,” and “patient” refer to any individual who is the target of treatment using the disclosed compositions. The subjects can be symptomatic or asymptomatic. The term does not denote a particular age or sex. A subject can include a control subject or a test subject. Typical subjects can include animals (e.g., mammals, such as mice, rats, rabbits, non-human primates, and humans).

### II. HDAC-G9a Dual Inhibitor Compounds

Dual inhibitor compounds of Formulae I, II, or II, and methods of making and using thereof, are described herein.

In some forms, the dual inhibitor compounds are defined according to Formula I:

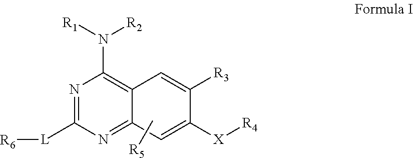

where X is absent or oxygen (O), nitrogen (NH or NR₁₈) or sulfur (S);

where R₁ is hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or one of the moieties:

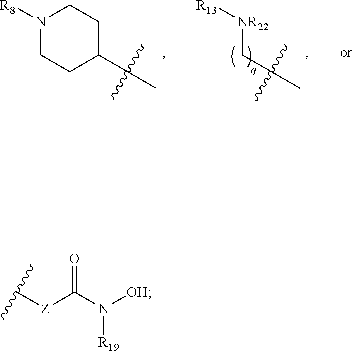

where q is an integer value in the range of 1-15, more preferably 1-10, most preferably 1-5;

where R₄, R₆, R₈, and R₁₃ are independently hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

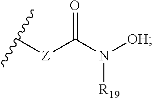

where Z is absent or a linking moiety, wherein the linking moiety is oxygen (O), nitrogen (NR₂₃), sulfur (S), optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where L is absent or a linking moiety, where the linking moiety is optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and

where R₂, R₃, R₅, R₁₈, R₁₉, R₂₂, and R₂₃ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and

where at least one of R₁, R₄, R₆, R₈, or R₁₃ is the moiety:

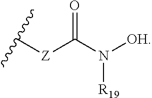

In certain forms of compounds according to Formula I, Z is:

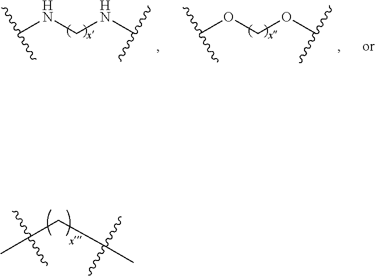

where x′, x″, and x′″ are integer values independently in the range of 1-15, more preferably 1-10, most preferably 1-5.

In certain other forms of compounds according to Formula I, Z is absent and R₆ is:

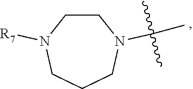

where R₇ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and the remaining groups/variables are as previously defined.

In other forms, the dual inhibitor compounds are defined according to Formula II:

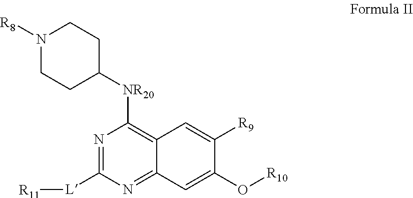

where R₈, R₁₀, and R₁₁ are independently hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

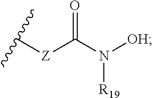

where Z is absent or a linking moiety, where the linking moiety is oxygen (O), nitrogen (NR₂₃), sulfur (S), optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where L′ is absent or a linking moiety, where the linking moiety is optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and

where R₉, R₂₀, R₂₃, and R₁₉ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and

where at least one of R₈, R₁₀, or R₁₁ is the moiety:

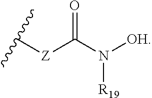

In some forms of compounds according to Formula II, R₈ is an optionally substituted benzyl.

In certain forms of compounds according to Formula II, Z is:

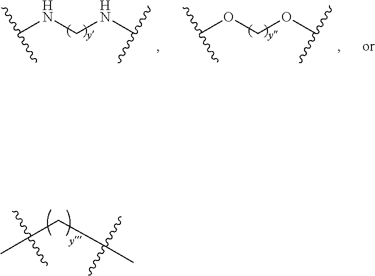

where y′, y″, and y′″ are an integer value in the range of 1-15, more preferably 1-10, most preferably 1-5.

In certain other forms of compounds according to Formula II, Z is:

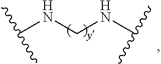

where y′ is as defined above;

R₁₁ is:

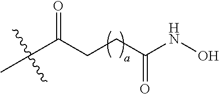

where a is an integer value in the range of 1-15, more preferably 1-10, most preferably 1-5; and

R₈, R₉, and R₁₀ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl. In preferred forms, R₈ and R₁₀ are an optionally substituted alkyl, optionally substituted aryl, or optionally substituted heteroaryl, and R₉ is hydrogen or an optionally substituted alkoxyl.

In yet other forms of compounds according to Formula II, Z is absent and R₁₁ is:

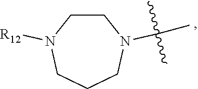

where R₁₂ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and the remaining groups/variables are as previously defined.

In certain forms of compounds according to Formula II, Z is absent, R_{H} is:

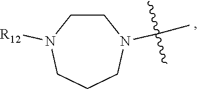

where R₁₂ is as previously defined;

R₈ is:

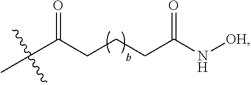

where b is an integer value in the range of 1-15, more preferably 1-10, most preferably 1-5; and

R₉ and R₁₀ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl. In preferred forms, R₉ is an optionally substituted alkoxyl and R₁₀ is an optionally substituted alkyl, optionally substituted aryl, or optionally substituted heteroaryl.

In yet other forms of compounds according to Formula II, Z is absent; R₁₁ is:

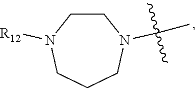

where R₁₂ is as previously defined;

R₁₀ is:

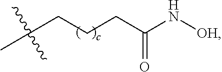

where c is an integer value in the range of 1-15, more preferably 1-10, most preferably 1-5; and

R₈ and R₉ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl. In preferred forms, R₈ is an optionally substituted alkyl, optionally substituted aryl, or optionally substituted heteroaryl and R₉ is an optionally substituted alkoxyl.

In still other forms, the dual inhibitor compounds are defined according to Formula III:

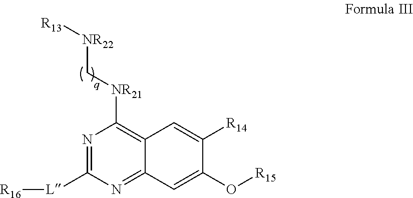

where q is an integer value in the range of 1-15, more preferably 1-10, most preferably 1-5;

where R₁₃, R₁₅, and R₁₆ are independently hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

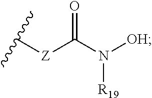

where Z is absent or a linking moiety, where the linking moiety is oxygen (O), nitrogen (NR₂₃), sulfur (S), optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where L″ is absent or a linking moiety, where the linking moiety is optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;

where R₁₄, R₂₁, R₂₂, R₂₃, and R₁₉ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and

where at least one of the R₁₃, R₁₅, or R₁₆ is the moiety:

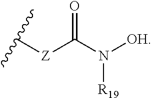

In certain forms of compounds according to Formula III, Z is:

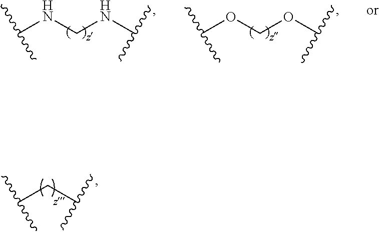

where z′, z″, and z′″ are an integer value in the range of 1-15, more preferably 1-10, most preferably 1-5.

In other forms of compounds according to Formula III, Z is absent and R₁₆ is:

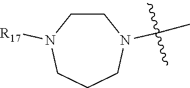

where R₁₇ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and the remaining groups/variables are as previously defined.

In certain forms of compounds according to Formula III, Z is absent;

R₁₆ is:

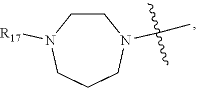

where R₁₇ is as previously defined; q is 2;

R₁₃ is:

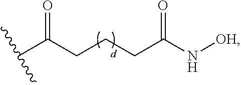

where d is an integer value in the range of 1-15, more preferably 1-10, most preferably 1-5; and

R₁₄ and R₁₅ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl. In preferred forms, R₁₄ is a hydrogen or an optionally substituted alkoxyl and R₁₅ is an optionally substituted alkyl, optionally substituted aryl, or optionally substituted heteroaryl.

The dual inhibitor compounds of Formulae I-III described above may have one or more chiral centers and thus exist as one or more stereoisomers. Such stereoisomers can exist as a single enantiomer, a mixture of diastereomers or a racemic mixture are encompassed by the present disclosure. As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

Non-limiting examples of HDAC-G9a dual inhibitors of Formulae I-III include, but are not limited, to the following exemplary compounds:

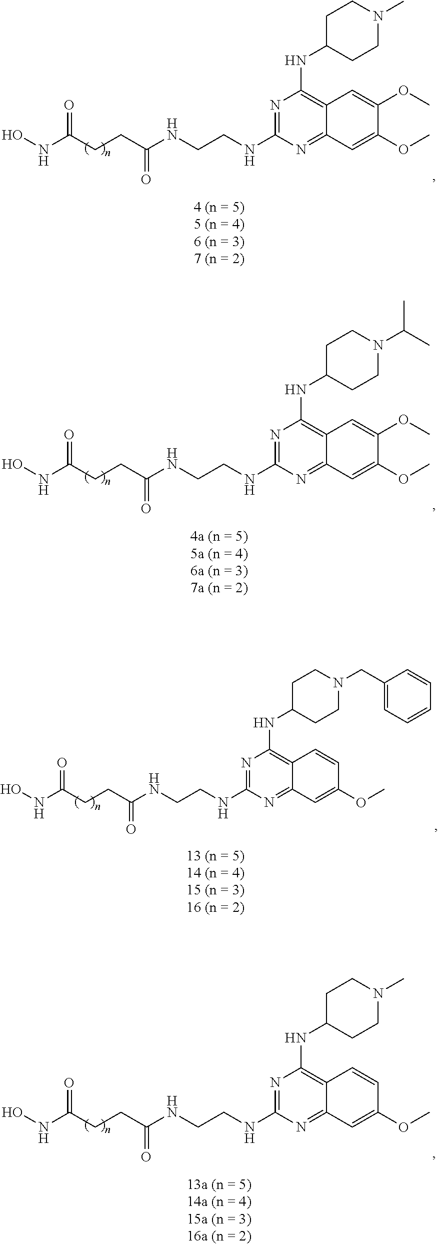

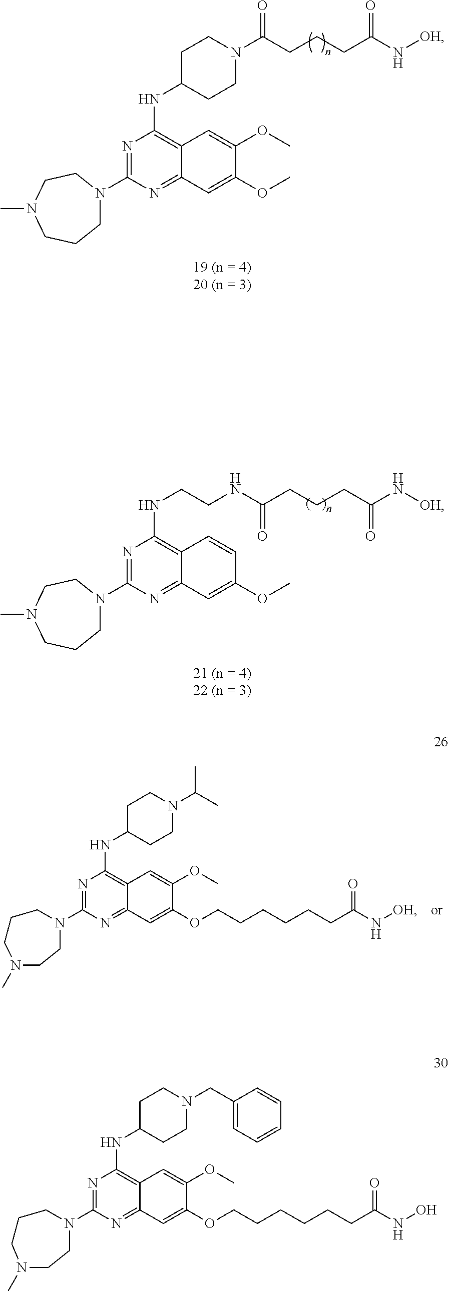

and pharmaceutically acceptable salts and solvates thereof.

### III. Formulations

Formulations containing one or more of the compounds described herein may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, PA: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

A. Other Active Agents

The HDAC-G9a dual inhibitor compounds described herein can be administered adjunctively with other active compounds. These compounds include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics. “Adjunctive administration”, as used herein, means the HDAC inhibitors can be administered in the same dosage form or in separate dosage forms with one or more other active agents.

Specific examples of compounds that can be adjunctively administered with the GDAC inhibitors include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko bilboa, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenytoin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof

1. Targeting to Cancer Cells or Tissue

The dual inhibitor compounds described herein can be bound to, or encapsulated within particles having on their surface, molecules that bind to antigens, ligands or receptors that are specific to cancer cells, tumor cells or tumor-associated neovasculature, or are upregulated in tumor cells or tumor-associated neovasculature compared to normal tissue, in order to target the drugs to the cancer cells or tissues thereof (i.e., tumors).

### IV. Methods of Preparation

The dual inhibitor compounds described herein can be made using conventional techniques known in art. Exemplary non-limiting methods of synthesizing dual inhibitor compounds are described in the Examples below (see Schemes I-V).

The dual inhibitor compounds produced according to the methods and reactions described may be recovered, obtained, isolated, extracted, purified, crystallized, or separated by conventional methods known to those of skill in the art.

The dual inhibition activity of the compounds can be determined, for example, using screening assays of dual inhibitor compounds. Generally, compounds can be tested in an assay for one activity. Those compounds that exhibit this activity can then be tested in an assay for the other activity. Alternatively, the assays may be used to screen particular classes of compounds for HDAC and/or G9a inhibition properties and toxicity properties. Exemplary, but non-limiting, assays are described in the Examples.

It is expected that other compounds of Formulae I-III can be prepared using such art known methodologies.

### V. Methods of Using HDAC-G9a Dual Inhibitor Compounds

The dual inhibitor compounds described herein may be used as anti-cancer agents. Examples of cancer which may be treated include, but are not limited to, lung cancer, myeloma, leukemia, acute myeloid leukemia, carcinoma, hepatocellular carcinoma, lymphoma (such as, but not limited to, cutaneous T-cell lymphoma and peripheral T-cell lymphoma), breast cancer, prostate cancer, pancreatic cancer, cervical cancer, ovarian cancer, and liver cancer.

In some embodiments, the dual inhibitor compounds described herein can be used to treat diseases such as fungal infections, Alzheimer's disease, Huntington's disease, epilepsy, depression, inflammatory diseases, and HIV, all of which are affected by HDACs.

The compounds of general Formulae I-III and their pharmaceutically-acceptable addition salts, prodrugs, and/or solvates can also be used in the form of pharmaceutical formulations or compositions which facilitate bioavailability. One or more compounds of Formulae I-III may be administered in a single dosage form or in multiple dosage forms. Such preparations may be in solid form, for instance in the form of tablets, pills, capsules, or ampules or in liquid form, for example solutions, suspension, or emulsions. The preparations may be formulated for immediate release, delayed release, extended release, pulsatile release, and combinations thereof.

Pharmaceutical formulations or compositions in the form suitable for injection are subjected to conventional pharmaceutical operations such as sterilization and/or may contain adjuvants including, but not limited to, preservatives, stabilizers, wetting or emulsifying agents, and buffers.

The formulations or compositions contain an effective amount of one or more HDAC-G9a dual inhibitors. The doses in which the HDAC-G9a dual inhibitors and their salts, prodrugs, or solvates can be administered may vary widely depending on the condition of the patient and the symptoms to be treated. One of ordinary skill in the art can readily determine the necessary dosage based on the condition of the patient and the disease to be treated.

### VI. Methods of Administration

In general, methods of administering inhibitor compounds as described herein are well known in the art. In particular, the routes of administration can include administration via a number of routes including, but not limited to: oral, intravenous, intraperitoneal, intramuscular, transdermal, subcutaneous, topical, sublingual, or rectal means. Such administration routes and appropriate formulations are generally known to those of skill in the art.

Any acceptable method known to one of ordinary skill in the art may be used to administer a formulation containing the dual inhibitors to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some forms, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The formulations may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain forms, the administration of the formulation may be designed so as to result in sequential exposures to the double duplex-forming oligonucleotides, and donor oligonucleotides, over a certain time period, for example, hours, days, weeks, months or years. This may be accomplished, for example, by repeated administrations of a formulation or by a sustained or controlled release delivery system in which the oliogonucleotides are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems which are suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some forms, the system may allow sustained or controlled release of the composition to occur, for example, through control of the diffusion or erosion/degradation rate of the formulation containing the oligonucleotides. In addition, a pump-based hardware delivery system may be used to deliver one or more forms.

Examples of systems in which release occurs in bursts include systems in which the composition is entrapped in liposomes which are encapsulated in a polymer matrix, the liposomes being sensitive to specific stimuli, e.g., temperature, pH, light or a degrading enzyme and systems in which the composition is encapsulated by an ionically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be in the form of pellets, or capsules.

Use of a long-term release implant may be particularly suitable in some forms. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

### EXAMPLES

### Example 1. Synthesis and Evaluation of HDAC-G9a Dual Inhibitors

Materials and Methods:

Reagents were purchased from commercial suppliers Sigma-Aldrich, Alfa Aesar, TCI, or Acros and were used without further purification unless otherwise indicated. Anhydrous solvents (e.g., DMF, DIPEA, MeOH, DCM) were purchased from Sigma-Aldrich and used directly. The reaction progress was monitored using silica gel 60 F254 thin layer chromatography plates (Merck EMD Millipore). Microwave reactions were performed using Initiator for organic synthesis. Column chromatography was performed on a Isolera one system using SNAP columns with KP-Sil silica or Zip Si columns with KP-Sil normal phase silica cartridges (unless otherwise stated). The nuclear magnetic resonance spectra were recorded on a 400 MHz spectrometer interfaced to a PC using Topspin 3.1. Solvents used were CDCl₃ and CD₃OD. Chemical shifts reported in ppm. Coupling constants, when reported, are reported in hertz (Hz). High-resolution mass spectra (HRMS) data were acquired using orbitrap elite mass spectrometer with an electrospray ionization (ESI) source. All the samples were ran under FT control at 600 000 resolution. All temperatures are reported in ° C. The purity of all final compounds were confirmed by RP-HPLC analysis, was >95% or mentioned in the synthetic procedure. Analytical high-performance liquid chromatography (HPLC) was performed using a Waters Agilent 1260 infinity, column used was Agilent eclipse plus C18 3.5 μM reverse phase 150 mm×4.6 mm chromatography column. Samples were detected using a wavelength of 254 nm. All samples were analyzed using acetonitrile (0.1% TFA):water (0.1% TFA) 5-60% over 30 min and a flow rate of 0.4 mL/min. Preparative HPLC was performed using the XBridge prep C18, 5 μM, 10×150 mm column and a flow rate of 1 mL/min.

Cloning, Protein Expression, and Purification:

Mouse histone methyltransferase G9a (969-1263) cDNA was amplified from the cDNA of BALB/c mouse thymus, and the fragment was sub-cloned into a vector with a 6His-sumo tag. The mouse G9a (mG9a) was expressed in Escherichia coli BL21 (DE3) by the addition of 1 mM isopropyl-1-thio-D-galactopyranoside (IPTG) and incubated overnight at 16° C.

The 6His-sumo mG9a (969-1263) protein was purified using the following procedure: harvested cell pellet was re-suspended in 20 mM Tris (pH 8.0), 500 mM NaCl, 0.1% β-mercaptoethanol, and 1 mM PMSF. Cells were lysed by sonicating for 15 seconds with 6 second intervals for a total time of 15 minutes on an ice bath. The supernatant of cell lysate was loaded onto a Ni⁺ affinity column (Invitrogen) then washed with buffer (20 mM Tris-HCl pH 8.0, 500 mM NaCl, 20 mM imidazole, 0.1% β-mercaptoethanol, and 1 mM PMSF). The 6His-sumo tag was cleaved from the column by adding Ubiquitin-like-specific protease 1 (ULP-1) at 4° C. for 12 hours. Wash buffer was then run through the Ni⁺ column again and the elution buffer collected. Subsequently, advanced protein purification was done by HiTrap Q HP sequential Superdex 200 10/300 GL. Elute of every step was analyzed by SDS PAGE, stained by Coomassie brilliant blue (CBB).

MALDI-TOF-MS:

The in vitro inhibition of G9a by the synthesized compounds were measured by MALDI-TOF mass spectrum (Bruker MALDI TOF/TOF Analyzer). 400 nM purified G9a, 5 μM synthesized histone H3(1-21) and 10 μM non-radioactive S-adenosyl methionine (Sigma) were added in reaction buffer (50 mM HEPES pH 8.0, 5 μg/ml BSA and 0.1% (3-Mercaptoethanol) with or without inhibitors (5 μM). The reaction was incubated at room temperature for 30 min, and stopped by TFA. 1 μL of the sample was mixed with CHCA matrix and m/z peaks were obtained at reflection positive mode. The results of mass spectrum were analyzed using the Bruker flex analysis software and data processing was carried out as described below.

MALDI-TOF based experiments were performed according to the protocol developed by Chang et al. (Nat. Struct. Mol. Biol. 2009, 16, 312-317). MALDI spectra were collected using Bruker flex control software and analyzed by flex analysis. After labelling each cluster peaks of H3K9Me0, H3K9Me1 and H3K9Me2 for all of the tested concentrations, area under the cluster (AUC) were extracted by using the same flex analysis software. % abundance of each peak was calculated by following formula:

A=% Abundance of (H3K9Me0)=area of H3K9Me0/(area of H3K9Me0+area of H3K9Me1+area of H3K9Me2)

B=% Abundance of (H3K9Me1)=area of H3K9Me1/(area of H3K9Me0+area of H3K9Me1+area of H3K9Me2)

C=% Abundance of (H3K9Me2)=area of H3K9Me2/(area of H3K9Me0+area of H3K9Me1+area of H3K9Me2).

This was repeated for each spectra (3 multiples for each samples).

G9a catalyze dimethylation of H3K9 and hence formation of H3K9Me2 was considered as the product formation and H3K9Me0 and H3K9Me1 is considered substrate not modified to the final product. Hence here % conversion to product is also C, from this to get the % maximal activity (% MA), C was compared to the % conversion when no inhibitor was used (D).

Finally % inhibition was found by subtracting % MA₍ᵢ₎ from 100. An average of 3 values were reported.

Cell-Based Assays:

Cell lines Information: MDA-MB-231 (breast cancer cell line), HCT-8 (Human colon cancer), MCF-7 (breast cancer cell line), A549 (human lung cancer cell line), K562 (human immortalized myelogenous leukemia cell line), Hela (human cervical cancer cell line), HEK293 (normal cell line).

Reagents: CCK-8, Trichostatin A and trypsin were purchased from Sigma.

Cell line: MDA-MB-231, A549 cell lines were grown at 37° C./5% CO₂ in Dulbecco's Modified Eagle's Medium (from Sigma) supplemented with 10% fetal bovine serum and 2% 200 mM L-glutamine and 0.5% antibiotic-antimycotic solution (from Sigma).

HCT-8, Hela, K562 cell lines were grown at 37° C./5% CO₂ in RPMI 1640 medium (Gibco) supplemented with 10% fetal bovine serum and 0.5% antibiotic-antimycotic solution.

MCF-7 cell line was grown at 37° C./5% CO₂ in Eagle's Minimum Essential Medium supplemented with 10% fetal bovine serum and 0.5% antibiotic-antimycotic solution.

HDAC Activity Assay:

The manual assay was developed by Thomas's group (Ciossek et al., Anal. Biochem. 2008, 372, 72-81). HeLa cells were seeded into white 96-well cell culture plates (corning costar 3596) at a density of 8000-10000 cells/well (total volume 81 μl culture medium) and incubated under standard cell culture conditions (37° C., 5% CO₂). After 24 h, 9 μl inhibitors with different concentration were added to the HeLa cells and incubation was continued for 3 h under cell culture conditions. After this treatment period, 10 μl of a 2 mM stock solution of the substrate Boc-K(Ac)-AMC was added into the 96 well plates with Hela cells and inhibitors. Cell culture plates were incubated under standard cell culture conditions for an additional 3 h before addition of 100 μl/well lysis/developer buffer mix (50 mM Tris-HCl, pH 8.0, 137 mM NaCl, 2.7 mM KCl, 1 mM MgCl₂, 1 vol % Nonidet-P40, 2.0 mg/ml trypsin, 10 μM TSA). After final incubation for 3 h under cell culture conditions, fluorescence was measured at excitation of k=355 nm and emission of k=460 nm on the Perkin-Elmer Wallac Victor V 1420 multilabel plate reader (Perkin-Elmer, Wellesley, USA). A549 and K562 cell lines used the same method, respectively. IC50s were calculated using GraphPad Prizm statistical package with sigmoidal variable slope dose response curve fit.

G9a H3K9me2 Cellular Assay:

Cells were seeded at 8000-10000 cells (100 μl) in black-walled 96-well plates (Thermo 165305) and exposed to various inhibitor concentrations for 48 h. After the incubation, the media was removed and 100 μl fixation and permeabilization solution (2% formaldehyde in PBS) for fixation was added for 30 min. And then use 200 μl 0.1% Triton X100 in PBS washing solution to wash (allow wash to shake on a plate shaker for 5 minutes). After five washes, cells were blocked for 1 h with 150 μl blocking buffer to each well (1% BSA in PBS) (allow blocking at RT with moderate shaking on a plate shaker). After 1 h, remove the blocking buffer from the blocking step and add primary antibody in blocking buffer to cover the bottom of each well. (Three out of four replicates were exposed to the primary H3K9me2 antibody, Abcam no. 1220 at 1/500 dilution in 1% BSA, PBS for overnight, one replicate was reserved for the background control (only blocking buffer). The wells were washed five times with 0.1% Tween 20 in PBS, then secondary IR800 conjugated antibody (LiCor) and cell tag 700 stain added for 1 h (incubate for 1 h with gentle shaking at RT, protect plate from light during incubation). After 5 wash with 0.1% Tween 20 in PBS, remove wash solution completely from wells. Turn the plate upside down and tap or blot gently on paper towels to remove traces of wash buffer. The plates were read on an Odyssey CL_{X} (LiCor) scanner at both 800 nm (H3K9me2 signal) and 700 nm (cell tag 700 stain signal) channels. IC₅₀s were calculated using GraphPad Prizm statistical package with sigmoidal variable slope dose response curve fit.

Toxicity Assay:

A549, MDA-MB-231, HCT-8, MCF-7, and HEK293 cells were seeded at 8000-10000 cells (100 μl) in white 96-well plates and pre-incubate the plate for 24 h under standard cell culture conditions, respectively. Then the cells were exposed to the different inhibitors with various concentrations for 72 h. Finally, 10 μl of CCK-8 kit solution was added to each well and incubated for 3-4 hours under standard cell culture conditions, and the 96 well plates were measured the absorbance at 450 nm using Perkin-Elmer Wallac Victor ³V 1420 multi label plate reader (Perkin-Elmer, Wellesley, USA). EC₅₀s were calculated using GraphPad Prizm statistical package with sigmoidal variable slope dose response curve fit.

Syntheses of HDAC-G9a Dual Inhibitors:

The compounds were synthesized from the commercially available 2,4-dichloro-6,7-dimethoxyquinazoline (1) for the dimethoxy analogs (Scheme I) and synthesis of monomethoxy analogs were produced according to Scheme II, starting from 2-amino-4-methoxybenzoic acid (8). Initially, only a few analogs of class IV in accordance to Scheme I were synthesized, as class IV was designed to assess the effectiveness of the HDAC substitution while opening the piperazine ring originally present at the prototype BIX-01294 (2-(hexahydro-4-methyl-1H-1,4-diazepin-1-yl)-6,7-dimethoxy-N-[1-(phenylmethyl)-4-piperidinyl]-4-quinazolinamine trihydrochloride); this particular class was also intended for the structure-activity relationship study of group R^{a}. The bulky seven member ring was replaced with an ethylene diamine in order to measure an optimum chain length for the maximal HDAC inhibition activity, various esters with different chain lengths (three to seven carbons) to produce compounds 4, 5, 6 and 7. To examine the effect of a bulky group on the heterocyclic ring at the C₄ position (corresponding to -L-R₆ in Formula I), an isopropyl group was introduced at the tertiary amine instead of the methyl group to produce the set of compounds 4a, 5a, 6a and 7a (Scheme I). While investigating the binding characteristics of known G9a/GLP inhibitors, it was determined that the C₆ methoxy group of quinaziline ring does not contribute significantly to ligand-receptor interactions. Therefore, the methoxy at position C₆ was eliminated to find a balance for HDAC inhibition activity. Based on this rationale, compounds in class II as per Scheme II (below) were designed. Compounds 13-16 have a 4-aminobenzyl piperidine at C₄, while compounds 13a-16a possess an methylpiperidin-4-amine. Compounds with the HDAC pharmacophore on the C₄ carbon of quinazoline core were termed class I, with analogs 19 and 20 retaining the C₆ methoxy group, and 21 and 22 lacking the C₆ methoxy group. Compounds 26 and 30 as shown in Scheme IV and Scheme V (both below) were classified as class III and class IIIA compounds. The classification of compounds into any one of Classes I-IV is discussed further below in the section on Structure-Activity Relationship (SAR) Studies of the dual inhibitors.

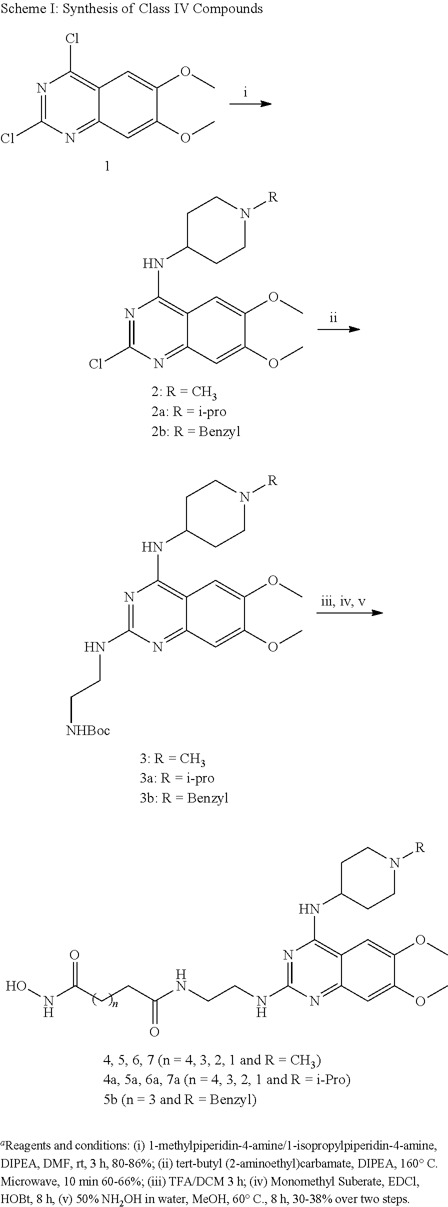

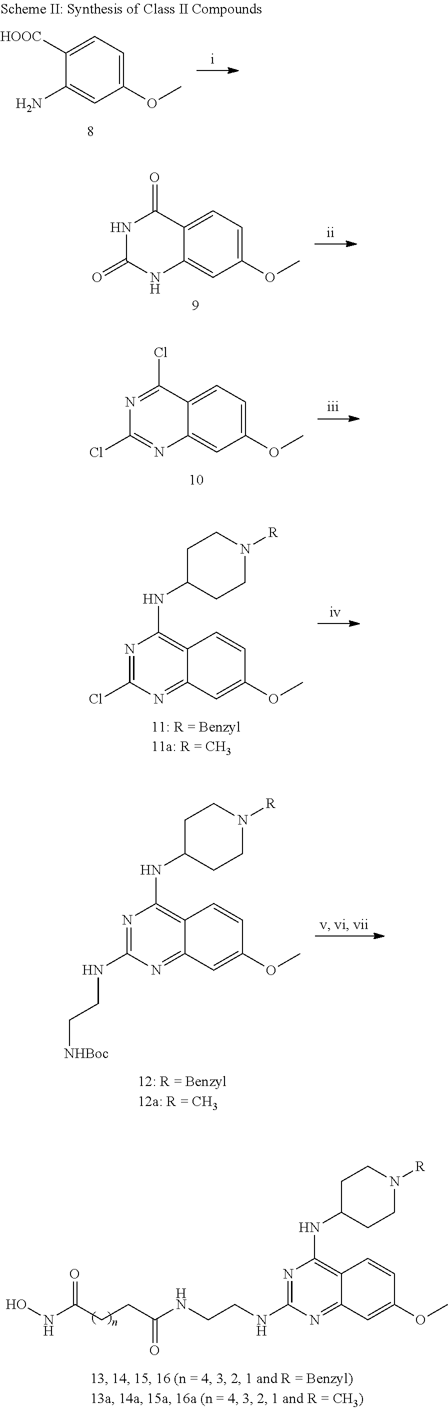

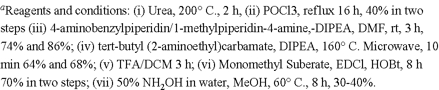

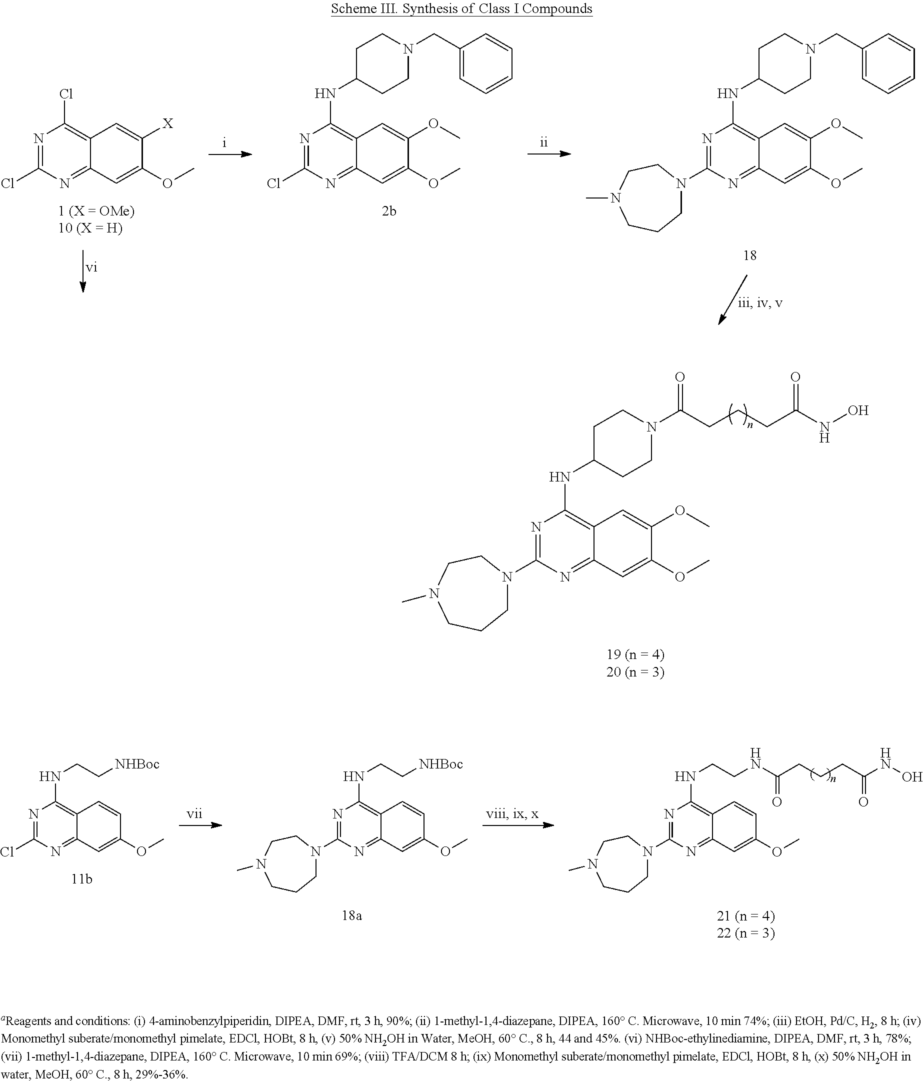

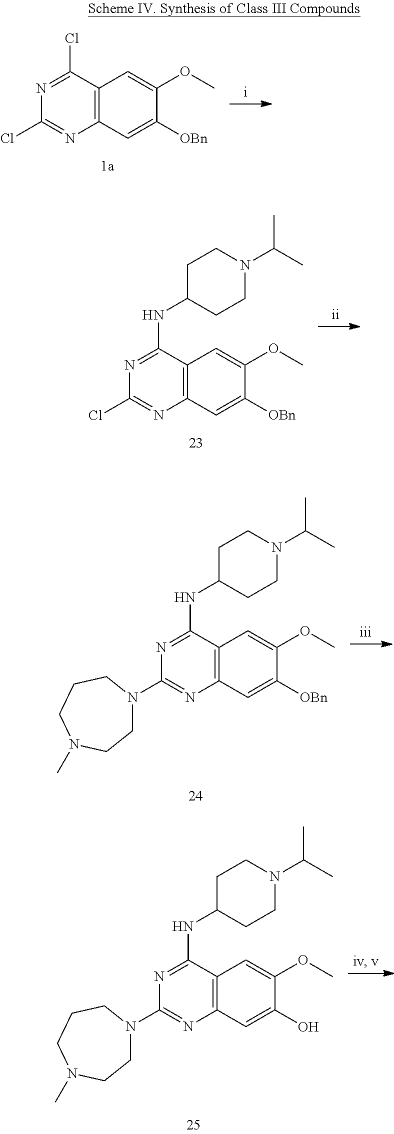

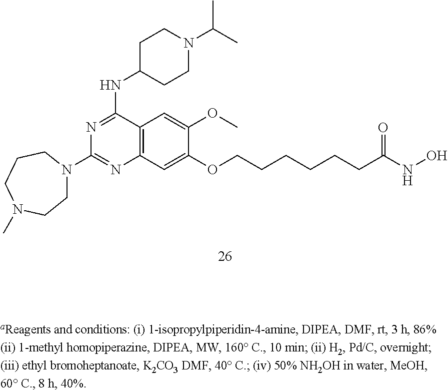 [SMILES: COc1cc2c(NC3CCN(C(C)C)CC3)nc(N3CCCN(C)CC3)nc2cc1OCCCCCCC(=O)NO]

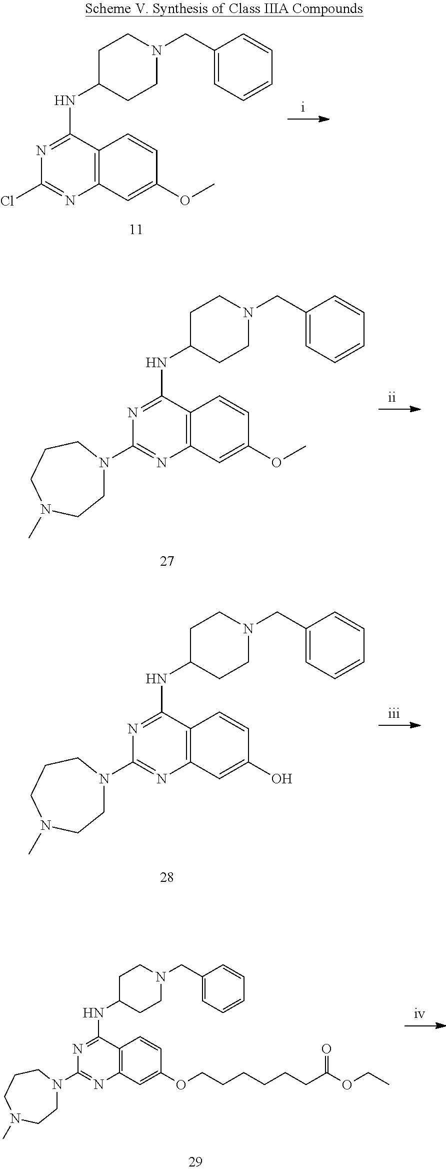 [SMILES: COc1ccc2c(NC3CCN(Cc4ccccc4)CC3)nc(Cl)nc2c1 ; COc1ccc2c(NC3CCN(Cc4ccccc4)CC3)nc(N3CCCN(C)CC3)nc2c1 ; CN1CCCN(c2nc(NC3CCN(Cc4ccccc4)CC3)c3ccc(O)cc3n2)CC1 ; CCOC(=O)CCCCCCOc1ccc2c(NC3CCN(Cc4ccccc4)CC3)nc(N3CCCN(C)CC3)nc2c1]

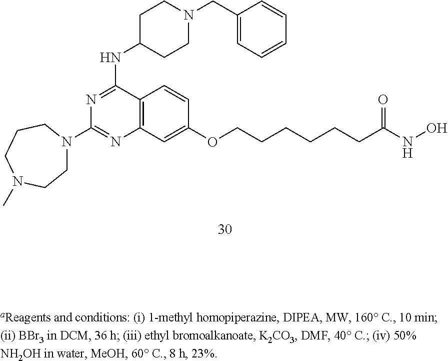 [SMILES: CN1CCCN(c2nc(NC3CCN(Cc4ccccc4)CC3)c3ccc(OCCCCCCC(=O)NO)cc3n2)CC1]

Scheme I compounds were synthesized from the commercially available starting material 1. An initial displacement reaction using a primary amine was used to introduce the C₄ selective substitution, with the second displacement to introduce the linker at the C₂ position following the microwave assisted reaction previously reported (Liu et al., J. Med. Chem. 2009, 52, 7950-7953). Boc-protected ethylene diamine was treated with compound 2 at 160° C. in microwave for 10 min to yield product 3 at excellent yield. Afterwards, deprotection of the amine 3 was performed with TFA/DCM, and the free amine was treated with corresponding monomethyl esters (carbon chain 2-6) in presence of coupling reagent EDCl and HOBt for about 8 hours to produce mono methyl ester substituted at the C2 position. This was further treated with hydroxylamine in water to get the corresponding hydroxamic acid derivatives, which were purified using reverse phase flash chromatography to obtain compounds 4-7 and 4a-7a in good yield. To synthesize the compounds in scheme II, the C₆ demethoxy core was required. This core was synthesized by cyclisation of 2-amino-4-methoxybenzoic acid in presence of urea at 200° C. without any solvent, yielding a crude solid residue after cooling, which was then suspended in water, filtered and dried to result in a 9 as a coffee brown powder. This was then dried and refluxed for 8 hours in POCl₃ to yield 10 (Van Horn et al., J. Med. Chem. 2014, 57, 5141-56). Appropriate displacement and coupling reactions on this core as demonstrated in the scheme II afforded compounds 13-17 and 13a-17a. Compounds with the HDAC pharmacophore at the C₄ position were synthesized from the starting material 1. The 1-benzylpiperidin-4-amine at C₄ was introduced by displacing chlorine, after which the second chlorine was displaced with 1-methyl-1,4-diazepane using microwave assisted reaction. Following this, Pd/C hydrogenolysis was used to eliminate the benzyl group and produce the free amine 18 for the coupling of monomethyl esters to result in compounds 19-22. Synthesis of compound 26 began from 4-hydroxy-3-methoxybenzonitrile and followed a previously reported procedure to produce compound 24 (Liu et al., J. Med. Chem. 2013, 56, 8931-8942). Afterwards, a nucleophilic substitution reaction with ethyl bromoheptanoate was used to introduce the linker, and later converted to the hydroxamic acid by treatment with hydroxylamine (50% H₂O) and methanol as solvent (Scheme IV). Compound 30 was also synthesized in a similar fashion, where BBr₃ was used to demethylate the C7 methoxy group of 27 (corresponding to —X—R₄ in Formula I) to release the free hydroxyl group as nucleophile (see Scheme V).

Synthetic Procedures and Compound Characterization of HDAC-G9a Dual Inhibitors:

H₃ (1-20, ARTKQTARKSTGGKAPRKQL, SEQ ID NO:1):

Peptide was synthesized through Fmoc-Strategy. Automated peptide synthesis was performed on Liberty Blue Peptide Synthesizer. Peptide were synthesized under microwave-assisted protocols on Wang resins. The deblock mixture was 20% piperidine in DMF. The following Fmoc-Lys(Boc)-Wang resin from Novabiochem were employed. The Fmoc protected amino acids were purchased from Chempep. Cocktail of TFA/TIS/Dodt/H₂O (92.5:2.5:2.5:2.5) was used to cleave peptides off the resin. After cleavage, crude peptide was purified through a reverse phase C18 column (purchased from Agilent, Eclipse XDB-C18, 5 μm, 9.4*250 mm).

Procedure A:

General procedure for compounds 2, 2a and 2b, 4-amino-piperidines (18.01 mmol) were added to a solution of 2,4-dichloro-6,7-dimethoxyquinazoline (2.11 g, 8.14 mmol in DMF 20 mL), followed by the addition of N,N-diisopropylethylamine (1.5 mL, 8.62 mmol) and the resulting mixture was stirred at room temperature for 2 hours until TLC showed that the starting material had disappeared. Water was added to the reaction mixture, and the resulting solution was extracted with ethyl acetate. The organic layer was washed with 0.5% acetic acid aqueous solution and brine, dried and concentrated to give the crude product, which was purified on flash column via eluting with hexane-ethyl acetate (20%) to get 3.0 g of the desired compound, yield 80-86%. Spectral properties of the product were matched with the reported compounds.

Procedure B:

General procedure for compounds 3, 3a, and 3b, Compound 2 (6.0 mmol) was dissolved in 8 mL of isopropanol. To this solution was added tert-butyl (2-aminoethyl)carbamate (1.92 g, 12 mmol) and DIPEA (1.5 mL, 7.2 mmol). The resulting solution was placed inside a microwave at 160° C. for 10 min. After cooling, TLC indicated the reaction was completed. Solvent was removed under reduced pressure, the residue was dissolved in DCM, washed with saturated NaHCO₃ solution. The combined organic phase was dried over Na₂SO₄ and concentrated under reduced pressure. The residue was purified on silica gel column, eluting with 5% MeOH in DCM (containing 0.5% Et₃N) to give 1.8 g of the Boc-protected amino compound as pale yellow solid. Yield 60-66%.

### N2-(2-aminoethyl)-6,7-dimethoxy-N4-(1-methylpiperidin-4-yl)quinazoline-2,4-diamine (3)

Brown solid, 1.8 g, 66% yield. ¹H NMR (400 MHz, CDCl₃) δ 7.10 (s, 1H), 7.06 (s, 1H), 6.19 (s, 1H), 4.33-4.20 (m, 1H), 3.91 (s, 6H), 3.17 (d, J=5.4 Hz, 2H), 2.93-2.75 (m, 4H), 2.50 (s, 2H), 2.30 (s, 3H), 2.14 (m, 4H), 1.78-1.60 (m, 2H), 1.42 (s, 9H). ¹³C NMR (100 MHz, CDCl₃) δ 159.5, 155.9, 154.8, 153.2, 148.9, 147.9, 107.1, 106.7, 101.3, 80.2, 56.6, 56.1, 54.5, 46.8, 45.0, 39.2, 30.4, 27.8. HRMS (ESI): m/z calcd for C₂₃H₃₆N₆O₄ [M+H]⁺, 461.2876; found, 461.2862.

### N2-(2-aminoethyl)-6,7-dimethoxy-N4-(1-isopropylpiperidin-4-yl)quinazoline-2,4-diamine (3a)

Brown solid, 1.7 g, 60% yield. ¹H NMR (400 MHz, CDCl₃) δ 7.23 (s, 1H), 7.19 (s, 1H), 4.13 (s, 1H), 3.84 (s, 6H), 3.58-3.44 (m, 4H), 3.39 (d, J=4.9 Hz, 2H), 3.32 (d, J=4.6 Hz, 2H), 2.96-2.83 (m, 2H), 2.11 (t, J=11.3 Hz, 2H), 2.01 (d, J=10.8 Hz, 2H), 1.74-1.59 (m, 2H), 1.40 (s, 9H), 1.01 (s, 6H). ¹³C NMR (100 MHz, CDCl₃) δ 165.1, 158.8, 156.3, 154.5, 145.6, 112.04, 108.9, 99.6, 80.8, 56.3, 55.9, 52.6, 49.9, 48.4, 41.4, 40.8, 31.7, 28.4. HRMS (ESI): m/z calcd for C₂₅H₄₀N₆O₄ [M+H]⁺, 489.3145; found, 489.3156.

### N2-(2-aminoethyl)-6,7-dimethoxy-N4-(1-benzylylpiperidin-4-yl)quinazoline-2,4-diamine (3b)

Brown solid, 2.1 g, 64% yield. ¹H NMR (400 MHz, CDCl₃) δ 7.36-7.14 (m, 5H), 6.79 (s, 1H), 6.58 (s, 1H), 6.01 (s, 2H), 5.44 (s, 1H), 4.11 (d, J=12.4 Hz, 1H), 3.84 (m, 6H), 3.52 (s, 2H), 3.40-3.37 (m, 2H), 3.32 (d, J=4.6 Hz, 2H), 2.93-2.84 (m, 2H), 2.11 (t, J=11.3 Hz, 2H), 2.01 (d, J=10.8 Hz, 2H), 1.75-1.59 (m, 2H), 1.38 (s, 9H). ¹³C NMR (100 MHz, CDCl₃) δ 165.1, 158.8, 156.3, 154.5, 145.6, 138.1, 129.2, 128.2, 127.1, 112.01, 108.9, 102.3, 78.9, 63.0, 56.3, 55.9, 52.6, 49.9, 48.4, 41.4, 40.8, 31.7, 28.3. HRMS (ESI): m/z calcd for C₂₉H₄₀N₆O₄ [M+H]⁺, 537.3189; found, 537.3165.

NH-Boc protection was removed to get the free amines of 3, 3a and 3b using TFA/DCM overnight, dried amine was used directly in next step without further purification.

Procedure C:

General procedure for compounds 4-7 and 4a-7a, To a stirred solution of corresponding monomethyl ester (0.25 mmol) in anhydrous CH₂Cl₂ (5 mL) was added EDCI (70 mg, 0.35 mmol) followed by HOBt (50 mg, 0.35 mmol) at 0° C. After 30 min, a solution of compound 3 (138 mg, 0.3 mmol) and DIEPA (0.1 mL, 0.5 mmol) in CH₂Cl₂ (2 mL) was added drop-wise at 0° C. The mixture was allowed to stir at rt and monitored by TLC. Upon completion, the organic layer was washed with saturated aqueous NaHCO₃ solution followed by brine. The organic extracts were dried over Na₂SO₄, filtered, and concentrated under reduced pressure. The crude product was purified by flash chromatography (MeOH/DCM up to 20%) to afford desired compounds as colorless oily liquid. HRMS (ESI): m/z calcd for C₂₇H₄₂N₆O₅ [M+H]⁺, 531.3295; found, 531.3279, This intermediate in methanol (2.5 mL) was added a solution of hydroxylamine (1 mL, 50% in water). The resulting solution was stirred for 3 h at 60° C. Then solvent was removed under vacuum and the crude residue purified by flash chromatography using reverse phase silica gel column using H₂O (0.1% HCOOH)/CH₃CN (0.1% HCOOH) as eluent (0-100%). This afforded the expected derivatives as a yellow/brown solid. 30-38% over 2 steps.

### N1-(2-((6,7-dimethoxy-4-((1-methylpiperidin-4-yl)amino)quinazolin-2-yl)amino)ethyl)-N8-hydroxyoctanediamide (4)

44 mg, 33% yield. ¹HNMR (400 MHz, MeOD) δ 7.81 (s, 1H), 7.59 (s, 1H), 6.91 (s, 1H), 4.68 (s, 1H), 3.93 (s, 6H), 3.64-3.47 (m, 5H), 3.23 (d, J=1.4 Hz, 4H), 3.14 (m, 1H), 2.90 (d, J=16.0 Hz, 3H), 2.36-2.28 (m, 2H), 2.21-2.07 (m, 5H), 1.64-1.49 (m, 4H), 1.30 (s, 4H). ¹³C NMR (100 MHz, MeOD) δ 183.7, 167.5, 148.4, 128.3, 124.8, 124.0, 117.1, 110.9, 104.0, 55.4, 54.7, 53.3, 52.8, 46.1, 42.1, 41.8, 38.2, 35.9, 32.3, 28.2, 25.4. HRMS (ESI): m/z calcd for C₂₆H₄₁N₇O₅ [M+H]⁺, 532.3247; found, 532.3248, HPLC purity 95.45%; t_{R}=14.004

### N1-hydroxy-N8-(2-((4-((1-isopropylpiperidin-4-yl)amino)-6,7-dimethoxyquinazolin-2-yl)amino)ethyl)octanediamide (4a)

49 mg, 35% yield. ¹HNMR (400 MHz, MeOD) δ 7.76 (s, 1H), 7.70 (s, 1H), 6.90 (s, 1H), 4.72 (s, 1H), 3.92 (s, 6H), 3.60 (m, 5H), 3.48-3.38 (m, 4H), 3.33 (dd, J=3.2, 1.6 Hz, 1H), 2.39-2.19 (m, 5H), 2.07 (t, J=7.1 Hz, 2H), 1.56 (d, J=5.5 Hz, 4H), 1.41-1.28 (m, 9H). ¹³C NMR (100 MHz, MeOD) δ 171.6, 166.3, 156.7, 153.1, 147.4, 147.2, 142.2, 135.7, 125.2, 124.7, 120.0, 117.3, 110.5, 104.1, 103.7, 57.9, 55.5, 45.0, 34.1, 32.1, 28.3, 25.0, 15.8. HRMS (ESI): m/z calcd for C₂₈H₄₅N₇O₅ [M+H]⁺, 560.3560; found, 560.3554. HPLC purity 95.12%; t_{R}=14.820.

### N1-(2-((6,7-dimethoxy-4-((1-methylpiperidin-4-yl)amino)quinazolin-2-yl)amino)ethyl)-N7-hydroxyheptanediamide (5)

38 mg, 30% yield. ¹HNMR (400 MHz, MeOD) δ 7.70 (s, 1H), 6.97 (s, 1H), 4.69 (s, 2H), 3.96 (s, 6H), 3.85 (s, 1H), 3.67-3.49 (m, 4H), 3.47 (d, J=5.7 Hz, 2H), 3.25 (d, J=13.5 Hz, 2H), 3.15 (d, J=7.4 Hz, 1H), 3.05-2.83 (m, 4H), 2.31 (d, J=11.1 Hz, 2H), 2.23-2.06 (m, 5H), 1.64-1.52 (m, 3H), 1.35 (d, J=6.9 Hz, 2H). ¹³C NMR (100 MHz, MeOD) δ 174.8, 170.0, 163.8, 156.6, 156.0, 153.4, 147.3, 112.0, 108.9, 99.6, 56.8, 53.6, 46.0, 40.5, 38.7, 37.4, 34.4, 30.4, 28.3, 25.2. HRMS (ESI): m/z calcd for C₂₅H₃₉N₇O₅ [M+H]⁺, 518.3091; found, 518.3080. HPLC purity 95.21%; t_{R}=14.402

### N1-hydroxy-N7-(2-((4-((1-isopropylpiperidin-4-yl)amino)-6,7-dimethoxyquinazolin-2-yl)amino)ethyl)heptanediamide (5a)

51 mg, 38% yield. ¹HNMR (400 MHz, MeOD) δ 7.75 (s, 1H), 7.61 (s, 1H), 6.94 (s, 1H), 4.73 (s, 1H), 3.94 (s, 6H), 3.61 (m, 5H), 3.48 (s, 2H), 3.42-3.23 (m, 3H), 2.39-2.21 (s, 5H), 2.09 (t, J=6.9 Hz, 3H), 1.60 (s, 4H), 1.38 (m, 8H). ¹³C NMR (100 MHz, MeOD) δ 170.8, 167.7, 164.5, 155.5, 154.2, 152.0, 147.3, 110.8, 104.2, 98.1, 55.4, 53.4, 52.6, 48.2, 47.1, 38.5, 37.1, 35.4, 31.9, 28.5, 25.6, 15.8. HRMS (ESI): m/z calcd for C₂₇H₄₃N₇O₅ [M+H]⁺, 546.3405; found, 546.3385. HPLC purity 93.80%; t_{R}=14.991.

### N1-(2-((4-((1-benzylpiperidin-4-yl)amino)-6,7-dimethoxyquinazolin-2-yl)amino)ethyl)-N7-hydroxyheptanediamide (5b)

55 mg, 37% yield. ¹H NMR (400 MHz, MeOD) δ 7.67 (d, J=1.9 Hz, 1H), 7.44 (dt, J=15.7, 7.9 Hz, 5H), 6.96 (s, 1H), 4.48 (s, 1H), 3.96 (dd, J=12.2, 3.3 Hz, 7H), 3.63 (s, 3H), 3.54-3.44 (m, 2H), 3.28 (d, J=11.8 Hz, 2H), 2.72 (s, 2H), 2.32-2.16 (m, 5H), 2.10 (t, J=7.1 Hz, 1H), 1.94 (d, J=13.8 Hz, 2H), 1.60 (ddd, J=15.4, 12.7, 7.5 Hz, 4H), 1.41-1.20 (m, 2H). 13C NMR (100 MHz, MeOD) δ 174.9, 171.4, 167.7, 159.4, 156.3, 156.0, 153.3, 147.4, 136.1, 130.6, 130.4, 129.1, 128.7, 128.6, 103.6, 98.4, 97.9, 60.5, 55.5, 51.1, 40.1, 38.3, 35.3, 28.5, 27.9, 25.0, 24.8. HRMS (ESI): m/z calcd for C₃₁H₄₃N₇O₅ [M+H]⁺, 594.3440; found, 594.3460. HPLC purity 96.20%; t_{R}=14.001.

### N1-(2-((6,7-dimethoxy-4-((1-methylpiperidin-4-yl)amino)quinazolin-2-yl)amino)ethyl)-N6-hydroxyadipamide (6)

44 mg, 35% yield. ¹H NMR (400 MHz, MeOD) δ 7.65 (s, 1H), 6.93 (s, 1H), 4.69 (s, 1H), 3.95 (s, 6H), 3.60 (d, J=14.8 Hz, 4H), 3.21 (m, 3H), 2.89 (d, J=9.2 Hz, 4H), 2.29 (m, 4H), 2.13 (s, 3H), 1.90 (dt, J=13.6, 6.7 Hz, 1H), 1.64 (s, 4H), 1.39 (d, J=6.6 Hz, 2H). ¹³C NMR (100 MHz, MeOD) δ 170.8, 168.2, 159.3, 156.5, 156.2, 153.3, 147.3, 111.2, 108.6, 103.0, 55.5, 52.9, 46.0, 41.8, 40.56, 38.2, 37.4, 31.8, 28.2, 24.6. HRMS (ESI): m/z calcd for C₂₄H₃₇N₇O₅ [M+H]⁺, 504.2934; found, 504.2911. HPLC purity 96.81%; t_{R}=13.374

### N1-hydroxy-N6-(2-((4-((1-isopropylpiperidin-4-yl)amino)-6,7-dimethoxyquinazolin-2-yl)amino)ethyl)adipamide (6a)

46 mg, 35% yield. ¹HNMR (400 MHz, MeOD) δ 7.77 (s, 1H), 7.64 (s, 1H), 7.32 (s, 1H), 6.96 (s, 1H), 4.73 (s, 1H), 3.96 (s, 6H), 3.72-3.52 (m, 5H), 3.47 (s, 2H), 3.37 (d, J=15.2 Hz, 2H), 2.39 (d, J=12.1 Hz, 2H), 2.17 (m, 6H), 1.63 (s, 4H), 1.43 (d, J=6.3 Hz, 6H). ¹³C NMR (100 MHz, MeOD) δ 174.5, 170.5, 168.0, 157.1, 156.0, 153.5, 147.3, 113.4, 108.4, 89.7, 57.2, 55.8, 49.2, 49.5, 40.8, 39.7, 38.0, 32.5, 28.2, 24.7, 15.5. HRMS (ESI): m/z calcd for C₂₆H₄₁N₇O₅ [M+H]⁺, 532.3247; found, 532.3245. HPLC purity 96.95%; t_{R}=14.164

### N1-(2-((6,7-dimethoxy-4-((1-methylpiperidin-4-yl)amino)quinazolin-2-yl)amino)ethyl)-N5-hydroxyglutaramide (7)

40 mg, 33% yield. ¹H NMR (400 MHz, MeOD) δ 7.68 (s, 1H), 7.63 (s, 1H), 7.30 (s, 1H), 6.84 (s, 1H), 4.66 (s, 1H), 3.92 (s, 6H), 3.61 (s, 4H), 3.46 (s, 2H), 3.35 (d, J=15.4 Hz, 3H), 2.90 (m, 3H), 2.29 (m, 4H), 2.14 (d, J=6.6 Hz, 4H), 1.91 (s, 2H). ¹³C NMR (100 MHz, MeOD) δ 170.8, 168.2, 159.4, 159.3, 156.5, 153.3, 147.3, 136.1, 122.1, 111.2, 108.6, 103.7, 55.5, 52.9, 46.1, 41.8, 40.0, 38.4, 38.2, 35.2, 31.8, 28.2, 24.6. HRMS (ESI): m/z calcd for C₂₃H₃₅N₇O₅ [M+H]⁺, 490.2778; found, 490.2756. HPLC purity 95.61%; t_{R}=12.751

### N1-hydroxy-N5-(2-((4-((1-isopropylpiperidin-4-yl)amino)-6,7-dimethoxyquinazolin-2-yl)amino)ethyl)glutaramide (7a)

50 mg, 38% yield. ¹HNMR (400 MHz, MeOD) δ 7.67 (s, 1H), 6.93 (s, 1H), 4.72 (s, 2H), 3.94 (s, 6H), 3.59 (d, J=12.4 Hz, 5H), 3.47 (s, 2H), 3.36 (d, J=13.9 Hz, 2H), 2.37 (s, 2H), 2.22-2.12 (m, 6H), 1.63 (m, 4H), 1.44 (s, 6H). ¹³C NMR (100 MHz, MeOD) δ 173.1, 168.0, 158.5, 156.5, 153.3, 134.9, 147.5, 110.6, 103.7, 98.5, 57.8, 55.2, 40.1, 38.4, 35.7, 32.4, 31.7, 28.5, 16.2, 15.8. HRMS (ESI): m/z calcd for C₂₅H₃₉N₇O₅ [M+H]⁺, 518.3091; found, 518.3139. HPLC purity 93.38%; t_{R}=13.670.

Compounds 13-16 and 13a-16a:

### 2,4-dichloro-7-methoxyquinazoline

Compound 10 was prepared according to the previously reported procedure (Van Horn et al., J. Med. Chem. 2014, 57, 5141-56).

3.4 g of anthranilic acid (20 mmol) and 3.5 equiv of urea were finely powdered using mortar and pestle and heated to 200° C. in a round-bottom flask open to the atmosphere. After 2 h, the mixture was cooled, triturated with water, and filtered to give the product as crude. Product was dried and used in next step directly. Molecular ion peak for C₉H₈N₂O₃ was found at 192.0773. Crude quinazoline-2,4-dione and 2.4 g of N,N-diethylaniline were mixed in 45 mL of phosphorus oxychloride, and the mixture was refluxed overnight under an argon atmosphere. The crude reaction mixture was concentrated, neutralized the excess of POCl₃ using NaHCO₃ and extracted to EA; dried on Na₂SO₄ and evaporated, purified using flash column, eluting at 20% of EA/Hexane. White fluffy powder, 1.82 g, 40% overall yield. HRMS (ESI): m/z calcd for C₉H₆Cl₂N₂ [M+H]⁺, 228.9935; found, 228.9934.

### N-(1-benzylpiperidin-4-yl)-2-chloro-7-methoxyquinazolin-4-amine (11) and 2-chloro-N-(1-isopropylpiperidin-4-yl)-7-methoxyquinazolin-4-amine (11a)

Compound 11 and 11a were prepared according to the procedure A, using 1-methylpiperidin-4-amine or 1-benzylpiperidin-4-amine. 11: Yellow powder, 74%. ¹H NMR (CDCl₃, 400 MHz) δ ppm 7.54 (d, J=9.0 Hz, 1H), 7.27-7.34 (m, 5H), 7.10 (d, J=2.4 Hz, 1H), 7.04 (dd, J1=9.0 Hz, J2=2.4 Hz, 1H), 5.61 (d, J=7.71 Hz, 1H), 4.23-4.33 (m, 1H), 3.88 (s, 3H), 3.57 (s, 2H), 2.91 (d, J=11.9 Hz, 2H), 2.24-2.30 (m, 2H), 2.08-2.13 (m, 2H), 1.59-1.69 (m, 2H). ¹³C NMR (CDCl₃, 100 MHz) δ ppm 163.7, 129.8, 158.3, 153.3, 137.7, 129.3, 128.3, 127.3, 122.0, 117.9, 107.2, 106.9, 62.9, 55.7, 52.0, 48.0, 31.9. HRMS (ESI): m/z calcd for C₂₁H₂₃ClN₄O [M+H]⁺, 383.1639; found, 383.1610.

11a: Yellow powder, 86%. ¹H NMR (400 MHz, CDCl₃) δ 7.56 (d, J=9.1 Hz, 1H), 7.13 (s, 1H), 7.07 (d, J=9.1 Hz, 1H), 4.28 (s, 1H), 3.92 (s, 3H), 2.88 (d, J=11.0 Hz, 2H), 2.35 (s, 3H), 2.26 (t, J=11.6 Hz, 2H), 2.17 (d, J=12.2 Hz, 2H), 1.86 (s, 1H), 1.66 (m, 2H). HRMS (ESI): m/z calcd for C₁₅H₁₉ClN₄O [M+H]⁺, 306.1247; found, 307.1323.

### N2-(2-aminoethyl)-N4-(1-benzylpiperidin-4-yl)-7-methoxyquinazoline-2,4-diamine (12) and N2-(2-aminoethyl)-7-methoxy-N4-(1-methylpiperidin-4-yl)quinazoline-2,4-diamine (12a)

Compounds 12 and 12a were Obtained Via Procedure B:

12: Brown solid, 1.94 g, 64%. ¹H NMR (400 MHz, CDCl₃) δ 8.75 (s, 2H), 8.49 (d, J=5.2 Hz, 1H), 7.96 (s, 1H), 7.37-7.14 (m, 4H), 6.62 (d, J=8.9 Hz, 2H), 5.56 (s, 1H), 4.22 (s, 1H), 4.06-3.88 (m, 1H), 3.67 (d, J=10.9 Hz, 3H), 3.51 (d, J=19.7 Hz, 4H), 3.26 (s, 2H), 2.99 (dd, J=14.9, 7.4 Hz, 1H), 2.90 (s, 2H), 2.09 (d, J=35.0 Hz, 4H), 1.91 (d, J=12.4 Hz, 3H), 1.28 (d, J=10.5 Hz, 10H). HRMS (ESI): m/z calcd for C₂₈H₃₈N₆O₃ [M+H]⁺, 507.3084; found, 507.3047.

12a: Brown solid, 1.75 g, 68%. ¹H NMR (400 MHz, CDCl₃) δ 7.46 (d, J=8.9 Hz, 1H), 6.84 (s, 1H), 6.74 (d, J=8.8 Hz, 1H), 5.55 (s, 2H), 4.18 (s, 1H), 3.88 (s, 3H), 3.61 (d, J=3.9 Hz, 2H), 3.39 (d, J=4.8 Hz, 2H), 2.87 (d, J=11.2 Hz, 2H), 2.34 (s, 3H), 2.21 (t, J=11.3 Hz, 2H), 2.12 (d, J=11.8 Hz, 2H), 1.66 (dd, J=21.0, 10.4 Hz, 2H), 1.44 (s, 9H). HRMS (ESI): m/z calcd for C₂₂H₃₄N₆O₃ [M+H]⁺, 431.2771; found, 431.2767.

Compounds 13-17 and 13a-17a were synthesized according to procedure C from the corresponding free amines; Yield varied from 30-40%, yellow/brown solids were obtained after purification.

### N1-(2-((4-((1-benzylpiperidin-4-yl)amino)-7-methoxyquinazolin-2-yl)amino)ethyl)-N8-hydroxyoctanediamide(13)

45 mg, 31% yield. ¹H NMR (400 MHz, MeOD) δ 8.06 (s, 1H), 7.52 (d, J=33.6 Hz, 5H), 6.83 (d, J=31.2 Hz, 2H), 4.62 (s, 1H), 4.34 (s, 2H), 3.89 (s, 3H), 3.70-3.40 (m, 6H), 3.22-3.03 (m, 3H), 2.21 (m, 8H), 1.56 (s, 4H), 1.29 (s, 5H). ¹³C NMR (100 MHz, MeOD) δ 175.0, 171.5, 165.1, 159.8, 154.0, 151.3, 141.9, 130.9, 129.7, 129.6, 128.8, 125.4, 113.7, 102.9, 98.0, 59.9, 55.3, 50.7, 40.0, 38.3, 35.7, 32.2, 28.4, 28.3, 27.9, 25.4, 25.1. HRMS (ESI): m/z calcd for C₃₁H₄₃N₇O₄[M+H]⁺, 578.3455; found, 578.3444. HPLC purity 95.41%; t_{R}=16.756.

### N1-hydroxy-N7-(2-((7-methoxy-4-((1-methylpiperidin-4-yl)amino)quinazolin-2-yl)amino)ethyl)heptanediamide (13a)

44 mg, 33% yield. ¹H NMR (400 MHz, CDCl₃) δ 7.46 (s, 1H), 6.85-6.49 (m, 2H), 4.20 (s, 1H), 3.87 (s, 3H), 3.70-3.46 (m, 4H), 2.95 (d, J=10.6 Hz, 2H), 2.36 (s, 3H), 2.23 (dd, J=21.2, 12.2 Hz, 6H), 2.15-2.00 (m, 6H), 1.81 (d, J=10.6 Hz, 3H), 1.65 (s, 5H). ¹³C NMR (100 MHz, MeOD) δ 175.0, 168.6, 165.2, 153.8, 149.2, 134.8, 125.4, 113.7, 108.7, 102.9, 98.0, 55.2, 52.8, 46.2, 42.2, 40.0, 38.2, 35.6, 32.2, 28.4, 28.3, 28.1, 25.3, 25.0. HRMS (ESI): m/z calcd for C₂₅H₃₉N₇O₄ [M+H]⁺, 502.3142; found, 502.3143. HPLC purity 95.75%; t_{R}=13.767.

### N1-(2-((4-((1-benzylpiperidin-4-yl)amino)-7-methoxyquinazolin-2-yl)amino)ethyl)-N7-hydroxyheptanediamide (14)

49 mg, 35% yield. ¹H NMR (400 MHz, MeOD) δ 8.10 (d, J=9.1 Hz, 1H), 7.59-7.40 (m, 5H), 6.97 (d, J=9.1 Hz, 1H), 6.90 (s, 1H), 4.60 (s, 1H), 4.18 (s, 2H), 3.94 (s, 3H), 3.64 (d, J=8.0 Hz, 2H), 3.46 (dd, J=14.1, 8.1 Hz, 4H), 3.39-3.31 (m, 2H), 3.18-2.96 (m, 2H), 2.23 (dd, J=19.2, 11.7 Hz, 4H), 2.15-1.92 (m, 4H), 1.69-1.53 (m, 4H), 1.41-1.27 (m, 2H). ¹³C NMR (100 MHz, MeOD) δ 175.0, 171.4, 167.8, 165.3, 159.9, 154.0, 141.9, 131.2, 130.5, 129.1, 128.7, 125.3, 113.8, 103.0, 98.2, 60.5, 55.1, 51.0, 40.2, 38.2, 35.3, 32.0, 28.4, 28.0, 25.0, 24.8. HRMS (ESI): m/z calcd for C₃₀H₄₁N₇O₄[M+H]⁺, 564.3298; found, 564.3307. HPLC purity 95.02%; t_{R}=16.600.

### N1-hydroxy-N7-(2-((7-methoxy-4-((1-methylpiperidin-4-yl)amino)quinazolin-2yl)amino)ethyl) heptanediamide (14a)

49 mg, 40% yield. ¹HNMR (400 MHz, MeOD) δ 8.10 (s, 1H), 7.17 (s, 1H), 6.94 (m, 1H), 4.62 (d, J=62.4 Hz, 2H), 3.94 (s, 3H), 3.86 (s, 2H), 3.73-3.55 (m, 2H), 3.47 (s, 1H), 3.29 (m, 5H), 3.15 (dd, J=14.9, 7.5 Hz, 2H), 2.97-2.77 (m, 4H), 2.45-2.17 (m, 3H), 2.24-2.02 (m, 3H), 1.61 (s, 2H), 1.42-1.22 (m, 2H). ¹³C NMR (100 MHz, CDCl₃) δ 178.5, 174.4, 166.0, 165.6, 159.2, 153.1, 129.78, 117.7, 115.8, 106.9, 59.6, 53.5, 50.7, 46.8, 42.1, 39.7, 39.3, 36.0, 32.0, 29.8, 28.4. HRMS (ESI): m/z calcd for C₂₄H₃₇N₇O₄ [M+H]⁺, 487.2907; found, 488.2962. HPLC purity 94.16%; t_{R}=13.232.

### N1-(2-((4-((1-benzylpiperidin-4-yl)amino)-7-methoxyquinazolin-2-yl)amino)ethyl)-N6-hydroxyadipamide (15)

52 mg, 38% yield. ¹H NMR (400 MHz, MeOD) δ 8.03 (s, 1H), 7.52 (m, 6H), 6.78 (d, J=40.3 Hz, 2H), 4.61 (s, 1H), 4.36 (s, 2H), 3.87 (s, 3H), 3.60 (m, 4H), 3.46 (m, 2H), 3.38 (m, 3H), 2.49-1.78 (m, 8H), 1.64 (s, 4H). ¹³C NMR (100 MHz, MeOD) δ 171.25, 167.79, 165.02, 159.78, 153.88, 139.89, 131.05, 129.70, 129.50, 128.93, 113.70, 111.3, 103.5, 66.8, 59.84, 55.36, 50.73, 41.6, 39.2, 38.4, 35.38, 32.02, 27.80. HRMS (ESI): m/z calcd for C₂₄H₃₇N₇O₄ [M+H]⁺, 550.3142; found, 550.3148. HPLC purity 96.48%; t_{R}=16.262.

### N1-hydroxy-N6-(2-((7-methoxy-4-((1-methylpiperidin-4-yl)amino)quinazolin-2-yl)amino)ethyl)adipamide (15a)

41 mg, 35% yield. ¹H NMR (400 MHz, MeOD) δ 8.08 (s, 1H), 6.87 (d, J=33.0 Hz, 2H), 4.68 (s, 2H), 3.88 (d, J=22.3 Hz, 3H), 3.82-3.69 (m, 1H), 3.62 (s, 4H), 3.46 (s, 2H), 3.22 (m, 2H), 3.28-3.17 (m, 1H), 3.19-3.01 (m, 1H), 2.89 (d, J=15.2 Hz, 3H), 2.42-2.19 (m, 3H), 2.09 (d, J=29.5 Hz, 2H), 1.90 (s, 1H), 1.62 (s, 3H), 1.38 (d, J=6.5 Hz, 2H). ¹³C NMR (100 MHz, MeOD) δ 173.7, 170.0, 167.9, 164.5, 159.2, 153.4, 141.4, 124.6, 113.0, 102.3, 97.4, 54.4, 53.6, 52.2, 46.2, 45.5, 41.6, 39.3, 37.5, 34.3, 34.0, 27.5. HRMS (ESI): m/z calcd for C₂₃H₃₅N₇O₄ [M+H]⁺, 474.2829; found, 474.2807. HPLC purity 96.40%; t_{R}=12.879.

### N1-(2-((4-((1-benzylpiperidin-4-yl)amino)-7-methoxyquinazolin-2-yl)amino)ethyl)-N5-hydroxyglutaramide (16)

52 mg, 39% yield. ¹H NMR (400 MHz, MeOD) δ 8.01 (s, 1H), 7.51 (d, J=38.6 Hz, 5H), 6.76 (d, J=47.4 Hz, 2H), 4.49 (d, J=103.1 Hz, 3H), 3.86 (s, 3H), 3.53 (d, J=59.5 Hz, 5H), 3.22 (s, 3H), 2.23 (m, 6H), 1.93 (s, 4H). ¹³C NMR (100 MHz, MeOD) δ 174.1, 170.8, 167.9, 165.0, 159.7, 153.6, 141.7, 131.0, 129.0, 129.8, 128.3, 125.4, 115.4, 113.6, 102.8, 67.9, 59.9, 55.3, 50.7, 40.1, 38.3, 34.8, 31.7, 27.8, 21.6. HRMS (ESI): m/z calcd for C₂₃H₃₅N₇O₄ [M+H]⁺, 536.2985; found, 536.2998. HPLC purity 94.34%; t_{R}=16.051.

### N1-hydroxy-N5-(2-((7-methoxy-4-((1-methylpiperidin-4-yl)amino)quinazolin-2-yl)amino)ethyl)glutaramide (16a)

35 mg, 31% yield. ¹H NMR (400 MHz, MeOD) δ 8.10 (s, 1H), 6.87 (d, J=31.2 Hz, 2H), 4.70 (s, 1H), 3.92 (s, 3H), 3.73 (dd, J=12.9, 6.5 Hz, 1H), 3.66 (m, 3H), 3.46 (s, 2H), 3.35 (d, J=15.5 Hz, 2H), 3.23 (m, 1H), 2.88 (d, J=14.5 Hz, 2H), 2.46-2.21 (m, 4H), 2.15 (s, 2H), 1.90 (s, 2H), 1.37 (m, 4H). ¹³C NMR (100 MHz,) δ 175.8, 171.2, 166.0, 165.2, 153.3, 151.3, 127.6, 113.2, 110.1, 103.0, 57.8, 54.6, 50.5, 46.9, 41.8, 39.2, 36.4, 33.6, 31.4, 19.0. HRMS (ESI): m/z calcd for C₂₂H₃₃N₇O₄ [M+H]⁺, 460.2672; found, 460.2648. HPLC purity 95.90%; t_{R}=12.615.

### N-(1-benzylpiperidin-4-yl)-6,7-dimethoxy-2-(4-methyl-1,4-diazepan-1-yl)quinazolin-4-amine (18)

Compound 18 was synthesized according to the previously reported procedure (Liu et al., J. Med. Chem. 2009, 52, 7950-7953), which was treated with Pd/C under H₂ gas to get the free amine; HRMS (ESI): m/z calcd for C₁₁H₃₂N₆O₂ [M+H]⁺, 401.2264; found, 401.2642. This amine was directly used in procedure C while using monomethyl suberate ester to get 19 and monomethyl pimelate to obtain 20.

### 8-(4-((6,7-dimethoxy-2-(4-methyl-1,4-diazepan-1-yl)quinazolin-4-yl)amino)piperidin-1-yl)-N-hydroxy-8-oxooctanamide (19)

48 mg, 34% yield over 2 steps. ¹H NMR (400 MHz, MeOD) δ 7.68 (s, 1H), 7.20 (s, 1H), 4.66 (d, J=12.0 Hz, 1H), 4.51 (s, 1H), 4.22 (s, 2H), 4.12 (d, J=12.7 Hz, 1H), 3.96 (m, 8H), 3.45 (s, 2H), 3.28 (d, J=19.7 Hz, 3H), 2.83 (d, J=17.7 Hz, 4H), 2.47 (dd, J=15.0, 7.3 Hz, 2H), 2.36 (s, 2H), 2.16 (m, 4H), 1.65 (m, 6H), 1.40 (s, 5H). ¹³C NMR (101 MHz, MeOD) δ 172.6, 171.5, 167.5, 158.5, 155.8, 152.9, 147.6, 103.4, 102.7, 99.6, 56.3, 55.5, 55.4, 44.7, 43.7, 42.7, 40.7, 32.4, 32.2, 31.5, 30.6, 28.5, 28.3, 25.1, 25.0, 24.3. HRMS (ESI): m/z calcd for C₂₉H₄₅N₇O₅ [M+H]⁺, 572.3516; found, 572.3530. HPLC purity 94.71%; t_{R}=15.347.

### 7-(4-((6,7-dimethoxy-2-(4-methyl-1,4-diazepan-1-yl)quinazolin-4-yl)amino)piperidin-1-yl)-N-hydroxy-7-oxoheptanamide (20)

62 mg, 45% yield over two steps. ¹H NMR (400 MHz, MeOD) δ 7.72 (s, 1H), 7.23 (s, 1H), 4.66 (d, J=12.1 Hz, 1H), 4.52 (s, 1H), 4.26 (s, 2H), 4.12 (d, J=12.0 Hz, 2H), 3.96 (d, J=14.8 Hz, 8H), 3.49 (d, J=39.9 Hz, 4H), 2.86 (dd, J=25.3, 14.7 Hz, 4H), 2.64-2.29 (m, 4H), 2.27-1.92 (m, 4H), 1.83-1.50 (m, 6H), 1.50-1.34 (m, 2H). ¹³C NMR (100 MHz, MeOD) δ 172.5, 171.4, 167.2, 158.5, 155.9, 152.5, 147.7, 137.0, 103.5, 102.7, 99.2, 56.18, 55.6, 55.5, 49.4, 45.9, 44.6, 43.5, 42.4, 40.2, 32.4, 32.1, 31.4, 30.6, 28.2, 25.0, 24.8, 24.0. HRMS (ESI): m/z calcd for C₂₈H₄₃N₇O₅ [M+H]⁺, 558.3404; found, 558.3387. HPLC purity 96.04%; t_{R}=14.311.

Compounds 21 and 22:

Compound 10 was treated with NHBoc ethylinediamine as per the procedure A to get the intermediate 11b; which was further treated with 1-methyl-1,4-diazepane in accordance to procedure B to yield 18a, followed by Procedure C using; monomethyl suberate or monomethylpimelate to get 21 and 22.

### tert-butyl(2-((2-chloro-7-methoxyquinazolin-4-yl)amino)ethyl) carbamate (11b)

78% yield. ¹H NMR (400 MHz, CDCl₃) δ 7.70 (d, J=9.0 Hz, 2H), 7.08-6.88 (m, 2H), 5.39 (s, 1H), 3.85 (s, 3H), 3.67 (d, J=3.9 Hz, 2H), 3.57-3.37 (m, 2H), 1.40 (s, 9H). HRMS (ESI): m/z calcd for C₁₆H₂₁ClN₄O₃+[M+H]⁺, 353.1380; found, 353.1372.

### tert-butyl(2-((7-methoxy-2-(4-methyl-1,4-diazepan-1-yl)quinazolin-4-yl)amino)ethyl) carbamate (18a)

69% yield. ¹H NMR (400 MHz, CDCl₃) δ 7.56 (d, J=8.9 Hz, 1H), 6.95 (s, 1H), 6.85 (s, 1H), 6.63 (s, 1H), 5.52 (s, 1H), 4.04-3.93 (m, 2H), 3.86 (s, 3H), 3.62 (d, J=4.9 Hz, 2H), 3.44 (d, J=4.5 Hz, 3H), 2.73 (s, 2H), 2.69-2.51 (m, 2H), 2.37 (s, 3H), 2.03 (s, 2H), 1.41 (s, 9H). HRMS (ESI): m/z calcd for C₂₂H₃₄N₆O₃ [M+H]⁺, 431.2771; found, 431.2746.

### N1-hydroxy-N8-(2-((7-methoxy-2-(4-methyl-1,4-diazepan-1-yl)quinazolin-4 yl)amino)ethyl)octanediamide (21)

36 mg, 29% yield over 2 steps. ¹H NMR (400 MHz, MeOD) δ 7.97 (d, J=8.7 Hz, 1H), 7.16 (s, 1H), 7.02 (d, J=8.6 Hz, 1H), 4.28 (s, 2H), 3.94 (s, 3H), 3.87 (s, 2H), 3.74 (d, J=5.6 Hz, 3H), 3.60-3.48 (m, 4H), 3.42 (s, 2H), 2.89 (d, J=7.3 Hz, 3H), 2.39 (s, 2H), 2.21 (t, J=7.0 Hz, 4H), 1.55 (m, 4H), 1.29 (s, 4H). ¹³C NMR (100 MHz, MeOD) δ 175.5, 167.49 164.98 159.76 153.4, 124.9, 114.1, 103.6, 99.8, 55.3, 55.1, 48.2, 48.0, 47.8, 47.6, 47.4, 47.2, 46.9, 46.0, 43.5, 42.6, 41.3, 37.5, 35.6, 34.0, 28.5, 28.4, 25.4, 24.7, 24.0. HRMS (ESI): m/z calcd for C₂₅H₃₉N₇O₄ [M+H]⁺, 502.3142; found, 502.3128. HPLC purity 96.21%; t_{R}=13.810.

### N1-hydroxy-N7-(2-((7-methoxy-2-(4-methyl-1,4-diazepan-1-yl)quinazolin-4-yl)amino)ethyl)heptanediamide (22)

43 mg, 36% yield over 2 steps. ¹H NMR (400 MHz, MeOD) δ 7.98 (d, J=9.1 Hz, 1H), 7.14 (d, J=1.9 Hz, 1H), 7.00 (m, 1H), 4.27 (s, 2H), 3.93 (s, 5H), 3.88 (s, 1H), 3.88-3.66 (m, 4H), 3.52 (m, 5H), 3.42 (s, 2H), 2.90 (s, 3H), 2.39 (s, 1H), 2.24 (dd, J=10.9, 7.1 Hz, 5H), 1.84 (dd, J=13.7, 6.5 Hz, 2H). ¹³C NMR (100 MHz, MeOD) δ 174.99, 171.13, 168.17, 164.87, 159.67, 153.28, 125.03, 114.13, 103.56, 99.82, 55.19, 43.67, 42.61, 41.15, 37.62, 35.26, 31.88, 24.93, 24.60. HRMS (ESI): m/z calcd for C₂₄H₃₇N₇O₄ [M+H]⁺, 488.2985; found, 488.2960. HPLC purity 96.80%; t_{R}=13.370.

### N-hydroxy-8-((4-((1-isopropylpiperidin-4-yl)amino)-6-methoxy-2-(4-methyl-1,4-diazepan-1-yl)quinazolin-7-yl)oxy)octanamide (26)

7-(benzyloxy)-N-(1-isopropylpiperidin-4-yl)-6-methoxy-2-(4-methyl-1,4-diazepan-1-yl)quinazolin-4-amine (24) was synthesized from 1a, by following procedure A (23) and procedure B (24), then benzyl group was removed using Pd catalyzed hydrogenolysis; mixture of compound 24 (600 mg, 1.2 mmol) and 10 wt % Pd(OH)₂/C (90 mg) in ethanol (100 mL) was stirred for 40 hours at room temperature under hydrogen balloon. The reaction mixture was filtered and concentrated to provide the debenzylated product 4-((1-isopropylpiperidin-4-yl)amino)-6-methoxy-2-(4-methyl-1,4-diazepan-1-yl) quinazolin-7-ol (25) as brownish yellow solid, 90%.

### N-hydroxy-7-((4-((1-isopropylpiperidin-4-yl)amino)-6-methoxy-2-(4-methyl-1,4-diazepan-1-yl)quinazolin-7-yl)oxy)heptanamide (26)

Procedure D, Ethyl heptanoate (80 μL, 0.4 mmol) was added to the ice cold solution of compound 24 (200 mg, 0.4 mmol) in DMF and K₂CO₃ (280 mg, 2 mmol), reaction mixture was warmed to room temperature and then at 60° C. After 6 h reactions mixture was evaporated to get the residue and dissolved in DCM and washed with brine, organic layer was vacuum dried and eluted in flash column using reverse phase silica at 40% ACN/H₂O to get the intermediate ester; which was then dissolved in 2 mL of MeOH and treated with 50% NH₂OH/water mixture (1 mL) overnight to afford the targeted product. Reaction mixture was dried and purified using reverse column and further by HPLC using ACN (0.1% HCOOH)/H₂O (0.1% HCOOH) as eluent. 94 mg, 40% overall yield. ¹HNMR (400 MHz, MeOD) δ 7.61 (s, 1H), 7.06 (s, 1H), 4.14 (s, 3H), 3.97 (d, J=14.7 Hz, 6H), 3.50 (s, 4H), 3.15 (s, 3H), 3.05 (s, 3H), 2.74-2.57 (m, 3H), 2.38 (s, 3H), 2.22-2.03 (m, 3H), 1.90 (d, J=24.1 Hz, 3H), 1.63 (d, J=48.4 Hz, 4H), 1.48-1.42 (m, 3H), 1.40-1.31 (m, 6H). ¹³C NMR (100 MHz, MeOD) δ 170.9, 164.2, 158.0, 155.5, 152.0, 146.6, 110.9, 108.5, 101.5, 70.3, 57.4, 56.8, 56.6, 53.1, 46.4, 44.3, 32.4, 29.8, 29.2, 28.8, 26.8, 25.1, 24.1, 20.3. HRMS (ESI): m/z calcd for C₃₀H₄₀N₇O₄ [M+H]⁺, 572.3924; found, 572.3925. HPLC purity 96.80%; t_{R}=13.370.

### 8-((4-((1-benzylpiperidin-4-yl)amino)-2-(4-methyl-1,4-diazepan-1-yl)quinazolin-7-yl)oxy)-N-hydroxyoctanamide (30)

Targeted analog 30 was synthesized from the anthranillic acid starting material; 7-(benzyloxy)-N-(1-benzylpiperidin-4-yl)-2-(4-methyl-1,4-diazepan-1-yl)quinazolin-4-amine (27) prepared according to the procedure A and B. ¹HNMR (400 MHz, CDCl₃) δ 7.90 (d, J=3.6 Hz, 1H), 7.26-7.18 (m, 5H), 7.12 (s, 1H), 6.98 (d, J=3.9, 1H), 5.87 (s, 1H), 4.39-4.12 (m, 5H), 3.92 (s, 3H), 3.63 (s, 2H), 3.20 (d, J=4.6 Hz, 2H), 3.04 (d, J=2.4 Hz, 2H), 2.52 (d, J=2.4 Hz, 2H), 2.28 (s, 3H), 1.82-1.66 (m, 6H). ¹³C NMR (100 MHz, CDCl₃) δ 163.0, 159.0, 158.6, 138.4, 129.1, 128.2, 127.0, 122.2, 112.1, 104.8, 104.3, 63.1, 58.8, 57.2, 55.3, 52.4, 48.2, 46.6, 45.9, 45.8, 32.0, 27.6. MALDI-TOF: m/z for C₂₇H₃₆N₆O [M+H]⁺ is 461.9

### 4-((1-benzylpiperidin-4-yl)amino)-2-(4-methyl-1,4-diazepan-1-yl)quinazolin-7-ol (28)

Aryl demethylation using BBr3 was employed (McOmie et al., Tetrahedron 1968, 24, 2289-2292). BBr3 solution in DCM (1 ml, 1M) was added to the ice cold solution of compound 27 (450 mg, 1 mmol), resulting solution was allowed to be in normal rt and stirred the reaction mixture under inert atmosphere. Reaction was monitored using mass spec (MALDI-TOF); after completion of the reaction at about 48 h, water was added to the mixture and basified with NaHCO₃, extracted with DCM, washed with brine and dried to obtain a pale yellow solid, which was used for next steps without purification. MALDI-TOF: m/z for C₂₆H₃₄N₆O [M+H]⁺ is 447.8., ratio of product was over 90% to starting material.

### 8-((4-((1-benzylpiperidin-4-yl)amino)-2-(4-methyl-1,4-diazepan-1-yl)quinazolin-7-yl)oxy)-N-hydroxyoctanamide (30)

Procedure D using 28 as the starting material afforded the desired intermediate as colorless solid. HRMS (ESI): m/z calcd for C₃₅H₅₀N₆O₃ [M+H]⁺, 603.4022; found, 603.4031. Subsequently compound 29 was dissolved in 2 ml of MeOH and treated with 50% NH₂OH/water mixture (1 mL) overnight to afford the targeted product. Reaction mixture was dried and purified using reverse column and further by HPLC using ACN/H₂O as eluent. Fractions collected were concentrated and lyophilized to get brown powder. 54 mg, 23% yield over two steps. ¹H NMR (400 MHz, MeOD) δ 8.10 (d, J=9.2 Hz, 1H), 7.65-7.53 (m, 4H), 7.06 (d, J=2.2 Hz, 1H), 6.94 (d, J=8.9 Hz, 1H), 3.94 (d, J=19.0 Hz, 2H), 3.80 (s, 2H), 3.72-3.59 (m, 4H), 3.50 (s, 2H), 3.22 (s, 3H), 2.48 (s, 2H), 2.34 (d, J=14.2 Hz, 3H), 2.15 (s, 2H), 2.05 (s, 4H), 1.96 (s, 5H), 1.86 (s, 2H), 1.68 (s, 2H), 1.46 (s, 4H). ¹³C NMR (100 MHz, MeOD) δ 175.5, 167.4, 164.9, 159.7, 153.4, 140.3, 129.1, 128.2, 127.0, 124.9, 114.1, 109.0, 103.6, 68.7, 63.7, 55.3, 55.1, 46.0, 43.5, 42.6, 41.3, 34.0, 28.5, 28.4, 25.4, 24.7. HRMS (ESI): m/z calcd for C₃₃H₄₇N₇O₃ [M+H]⁺, 590.3819; found, 590.3832. HPLC purity 96.26%; t_{R}=14.192.

Structure Activity Relationship Studies of HDAC-G9a Dual Inhibitors:

A concurrent synthesis and testing strategy was used in establishing the primary structure-activity relationship (SAR), with efforts to examine the effect of introducing the HDAC pharmacophore to the quinazoline core and its subsequent impact on activity.

For the purposes of studying SAR, representative compounds were examined according to Classes I-IV according to the following non-limiting representation:

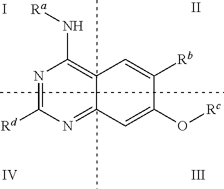

| R^{a} | R^{b} | R^{c} | R^{d} |
|---|---|---|---|
| 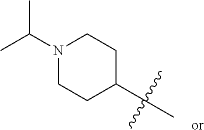 [SMILES: *C1CCN(C(C)C)CC1 ; "or"] | —OCH₃ | | |
| 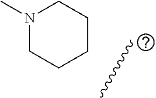 | | | |
| 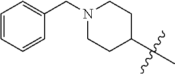 [SMILES: *C1CCN(Cc2ccccc2)CC1] | | —CH₃ | |
| 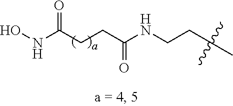 | —H | | 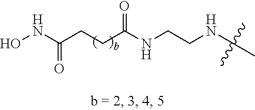 |
| 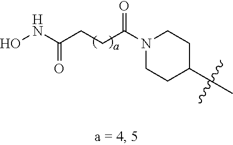 | | 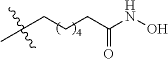 | 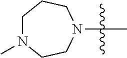 [SMILES: *N1CCCN(C)CC1] |

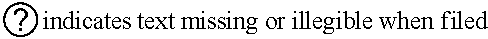

Classification into any of classes I-IV is based on the presence of a hydroxamic-containing substituent, such as those shown in the table above at the R^{a}, R^{b}, R^{c}, or R^{d} positions. For example, Class I compounds contain a hydroxamic-containing substituent at the R^{a} position, whereas Class III and IIIA compounds contain a hydroxamic-containing substituent at the R^{c} position. Class II compounds contain a hydroxamic-containing substituent at the R^{d} and R^{b} is a hydrogen, not a methoxy group. Class IV compounds contain a hydroxamic-containing substituent at the R^{d} position.

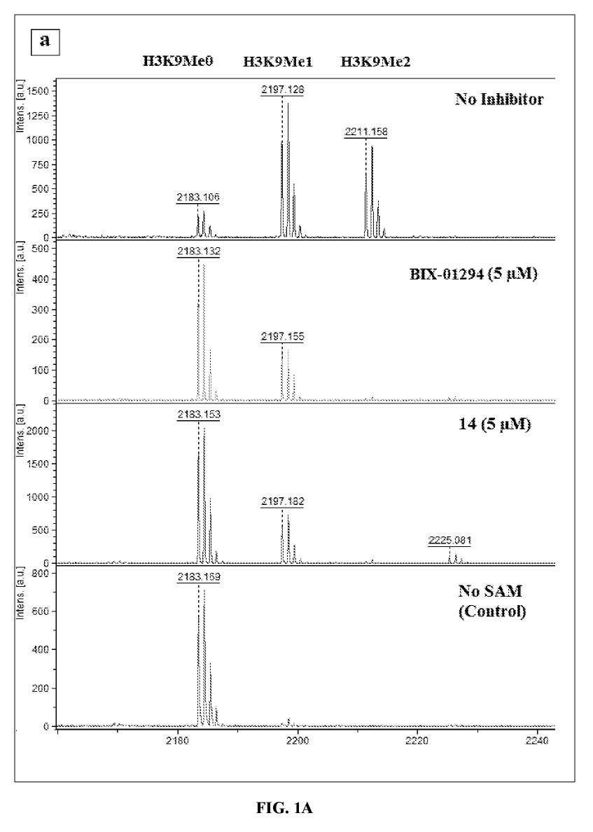

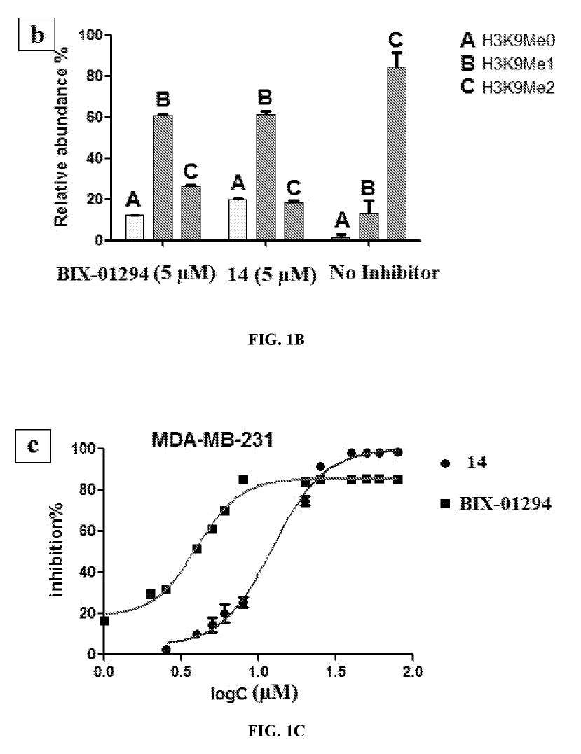

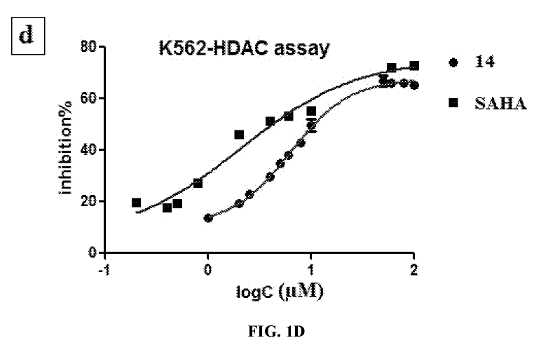

A biochemical assay using MALDI-TOF was used to visualize the effects of the synthesized compounds on G9a enzymatic activity. A biochemical reaction was carried out involving target enzyme G9a, methyl donor SAM and substrate H₃ peptide at a concentration of 400 nM, 10 μM and 5 μM respectively (Chang et al., Nat. Struct. Mol. Biol. 2009, 16, 312-317). After optimizing the reaction conditions and reaction time to obtain at least 80% conversion of the substrate to the methylated form (H3K9Me1 or H3K9Me2), but yet no tri-methylation, BIX-01294 was tested for an optimum level of inhibition and fixed the concentration as 5 μM for each inhibitor. A majority of the compounds retained G9a inhibition capabilities, indicated by the reduction in the ratio of the H3K9Me1 and H3K9Me2 peaks relative to the control reaction (see FIGS. 1A-1D and Table 1).

TABLE 1
MALDI-TOF methylation study of inhibitors
at 5 μM concentration for 30 min

| CPD# | Inhibition (%) | CPD# | Inhibition (%) |
|---|---|---|---|
| 4 | 26.96 ± 12.15 | 4a | 11.22 ± 3.68 |
| 5 | 65.02 ± 1.95 | 5a | 47.55 ± 9.32 |
| 6 | 26.64 ± 11.34 | 6a | 50.65 ± 16.51 |
| 7 | 19.58 ± 20.95 | 7a | 15.01 ± 15.73 |
| 13 | 62.00 ± 14.44 | 13a | 24.91 ± 17.42 |
| 14 | 69.55 ± 3.43 | 14a | 29.68 ± 23.32 |
| 15 | 47.85 ± 12.88 | 15a | 69.12 ± 24.54 |
| 16 | 40.32 ± 13.81 | 16a | 40.32 ± 13.81 |
| 19 | 10.71 ± 25.88 | 20 | 14.62 ± 15.81 |
| 21 | 22.75 ± 26.72 | 22 | 40.26 ± 15.33 |
| 26 | 35.61 ± 0.88 | 30 | 17.89 ± 5.51 |
| 5b | 38.01 ± 4.99 | BIX-01294 | 77.68 ± 5.73 |

These results corresponded to a MALDI-TOF study done in accordance to a previously reported procedures (Chang et al., Nat. Struct. Mol. Biol. 2009, 16, 312-317). With G9a inhibitory activity preserved, the effect of the compounds in cell were investigated, H3K9Me2 cell immunofluorescence in-cell Western (ICW) assays were used to assess G9a inhibition potential and homogeneous cellular histone deacetylases assay was used for assessing HDAC inhibition potential.

Functional Potency Evaluation for G9a Inhibition:

To assess the functional potency of the dual inhibitors, all compounds were evaluated by H3K9Me2 cell immunofluorescence in-cell Western (ICW) assays with the results shown in Table 2 (below). The MDA-MB-231 cell line was used in this study as this cell line possesses robust H3K9Me2 levels (Liu et al., J. Med. Chem. 2013, 56, 8931-8942). The results indicated that compounds belonging to class IV, having a hydroxamic acid-containing group at position R^{d} (southwest directing HDAC), exert a G9a inhibition activity comparable to the parent compound BIX-01294, with other compound classes, as defined above, being less potent.

TABLE 2
H3K9Me2 cell immunofluorescence in-cell Western
(ICW) assay results (MDA-MB-231 cell line)

| Compound | G9a IC₅₀ (μM) | Compound | G9a IC₅₀ (μM) |
|---|---|---|---|
| 4 | 96.69 ± 1.68 | 4a | 74.21 ± 1.94 |
| 5 | >100 | 5a | 66.63 ± 3.98 |
| 6 | >100 | 6a | >100 |
| 7 | 76.74 ± 0.89 | 7a | 54.55 ± 3.05 |
| 13 | 37.79 ± 2.80 | 13a | >100 |
| 14 | 7.136 ± 1.62 | 14a | 46.83 ± 1.97 |
| 15 | 72.10 ± 1.37 | 15a | 46.97 ± 3.33 |
| 16 | 90.26 ± 3.75 | 16a | 45.71 ± 1.76 |
| 19 | 99.63 ± 3.13 | 21 | ND |
| 20 | 97.51 ± 2.78 | 22 | 60.65 ± 3.66 |
| 26 | 27.37 ± 3.59 | 30 | >100 |
| BIX-01294 | 4.563 ± 1.2 | 5B | 87.39 ± 5.44 |

Functional Potency Evaluation for HDAC Inhibition:

Following confirmation of G9a inhibition activity, all of the synthesized compounds were tested for HDAC inhibition activity, as both targets are independent of each other. The enzymatic activity of HDAC was measured in intact cells using the homogeneous cellular assay method (Botrugno et al., Cancer Lett. 2009, 280, 134-44). Boc-K(Ac)-AMC was used as a cell-permeable HDAC substrate, as after deacetylation it is cleaved by trypsin to release the florescent 7-amino-4-methylcoumarin (AMC). The released AMC is proportional to the deacetylated substrate; therefore quantification was performed using fluorescence at excitation of k=355 nm and emission of k=460 nm. All compounds were tested in both Hela and K562 cell lines; compounds 13 and 14 showed significant HDAC inhibition with a comparable IC₅₀ to SAHA (N-hydroxy-N′-phenyl-octanediamide) (see Table 3 below).

TABLE 3
Cell based Homogenous HDAC assay results

| | | IC₅₀-HDAC | |
|---|---|---|---|
| entry | Hela^{c} | A549^{d} | K562^{e} |
| 4 | NA^{a} | NA | NA |
| 4a | NA | NA | NA |
| 5 | NA | NA | NA |
| 5a | NA | NA | NA |
| 6 | NA | NA | NA |
| 6a | NA | NA | NA |
| 7 | NA | NA | NA |
| 7a | NA | NA | NA |
| 13 | 15.33 ± 0.79 | >100 | 27.75 ± 0.59 |
| 13a | >100 | >100 | >100 |
| 14 | 13.80 ± 1.22 | >100 | 5.735 ± 1.23 |
| 14a | >100^{b} | >100 | >100 |
| 15 | >100 | >100 | >100 |
| 15a | >100 | >100 | >100 |
| 16 | >100 | >100 | >100 |
| 16a | >100 | >100 | >100 |
| 19 | >100 | >100 | >100 |
| 20 | >100 | >100 | >100 |
| 26 | >100 | NA | >100 |
| 30 | NA | NA | >100 |
| 21 | >100 | >100 | >100 |
| 22 | >100 | >100 | >100 |
| BIX | NA | NA | NA |
| SAHA | 5.044 ± 0.53 | >100 | 2.056 ± 0.59 |

NA^{a} not active up to the highest concentration tested (the highest concentration of all compounds is 100 uM;
>100^{b} in the cases where the IC50 did not reach at the highest tested concentration (100 uM);
^{c}Hela: human cervical cancer cell line;
^{d}A549: human lung cancer cell line;
^{e}K562: human immortalized myelogenous leukemia cell line;
SAHA was used as the positive control.
Data are shown as mean ± SD of triplicate.

An evaluation of the structures of compounds 13 and 14 indicated that the best HDAC inhibitory activity was displayed by class IV compounds, wherein R^{d} is a hydroxamic containing moiety, such as

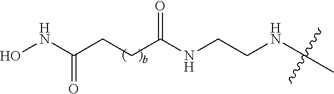

where b is 3 or 4. It was, however, not conclusive as to whether the R^{a} and R^{d} substitutions were responsible for the superior inhibitory activity observed. Examination of the tested compounds indicated that the best HDAC inhibitory activity was observed for compounds having a benzyl group at the 4-aminopiperidin ring (R^{a}) with the presence of a hydrogen atom instead of bulky methoxy group at the C₆ position of the quinazoline ring (W). To determine whether it was both R^{a} and R^{c} substitutions working in conjunction, or if only one was important for inhibition activity, the compound 5b was synthesized from 3b with a benzyl-containing substituent group at R^{a} and a methoxy at R^{b}. When this compound was tested for HDAC inhibition activity, the inhibition potential was much lower than that of 14, indicating both factors are responsible for the inhibition activity—an aromatic ring at R^{a} is very important for HDAC activity while a methoxy group at C₆ position of the quinazoline core greatly reduces HDAC inhibition. Compounds 11-14 were only different by the chain length; further testing indicated that 5 or 6 methylene groups are optimal for inhibition activity hence all further inhibitors were designed with these chain lengths. Compounds with the R^{c} substituted were also found to be poor inhibitors of HDAC, possibly due to either the bulky group at R^{a} or R^{b} or both (Cai et al., J. Med. Chem. 2010, 53, 2000-9). Similarly, compounds 15-18 with the R^{a} substituted with the HDAC chain gave low inhibition activity, leaving 13 and 14 as the best compounds.

Cell Anti-Proliferation Assay:

Cell anti-proliferation assays were performed to determine the toxicity of these inhibitors. Several cell lines (MDA-MB-231, MCF-7, A549 and HCT-8) were incubated and then treated with varying concentrations of the inhibitors for 72 h, respectively. After the first cell culture screening, it was determined that the inhibitors were more effective with breast cell lines (MDA-MB 231 and MCF-7) compared to other cell lines, particularly compound 13 and 14 (Table 4). These compounds were further evaluated against the control cell line HEK293 to test their toxicity with a non-cancerous cell line.

TABLE 4
Detailed results of cytotoxicity study

| | EC50(uM) | | | |
|---|---|---|---|---|
| Entry | MDA-MB-231^{c} | MCF-7^{d} | A549^{e} | HCT-8^{f} |
| 4 | >100^{b} | >100 | NA^{a} | NA |
| 4a | >100 | >100 | NA | NA |
| 5 | >100 | >100 | NA | NA |
| 5a | >100 | >100 | NA | NA |
| 6 | >100 | >100 | NA | NA |
| 6a | >100 | >100 | NA | NA |
| 7 | >100 | >100 | NA | NA |
| 7a | >100 | >100 | NA | NA |
| 13 | 89.33 ± 1.23 | 79.43 ± 2.72 | >100 | >100 |
| 13a | >100 | >100 | NA | >100 |
| 14 | 10.02 ± 1.66 | 37.36 ± 2.20 | 36.24 ± 1.76 | 73.07 ± 1.21 |
| 14a | 82.32 | >100 | NA | NA |
| 15 | 95.15 | >100 | NA | NA |
| 15a | 77.62 | >100 | NA | NA |
| 16 | 38.15 | 57.29 | >100 | 83.9 |
| 16a | 90.54 | >100 | NA | NA |
| 19 | >100 | >100 | NA | NA |
| 20 | >100 | >100 | NA | NA |
| 21 | >100 | >100 | NA | NA |
| 22 | >100 | >100 | NA | NA |
| 26 | 31.28 ± 3.30 | >100 | NA | NA |
| 30 | 24.01 ± 3.64 | >100 | NA | NA |
| 5b | 12.29 ± 3.27 | 74.57 ± 1.81 | NA | NA |
| BIX01294 | 2.155 ± 0.88 | 8.103 ± 1.99 | 21.74 ± 2.73 | |
| SAHA | 2.874 ± 0.84 | 8.124 ± 4.98 | 19.31 ± 1.26 | <10 |

NA^{a}, not active up to the highest concentration tested (the highest concentration of all compounds is 100 uM.
>100^{b} in the cases where the IC50 did not reach at the highest tested concentration (100 uM).
^{c}MDA-MB-231: breast cancer cell line;
^{d}MCF-7: breast cancer cell line;
^{e}A549: human lung cancer cell line;
^{f}HCT-8: Human colon cancer;
SAHA and BIX01294 are used as the positive controls;
Cells were exposed to the different inhibitors with various concentrations for 72 h, Inhibition of cell growth by the listed compounds was determined by using CCK-8 kit. Data are shown as mean ± SD of triplicate.

As seen in Table 4 (above), both SAHA and BIX-01294 appear to be toxic to cancer and normal cells, but compounds 13 and 14 displayed lower toxicity, particularly compound 14. Compound 14 also showed improved anti-proliferation abilities in all cancer cell lines and reduced toxicity in normal cell line compared to 13.

### DISCUSSION AND CONCLUSIONS

A combination of a G9a inhibitor and a HDAC inhibitor were tested in conjunction against MDA-MB-231 and MCF-7 cell lines treated with either SAHA (1-100 μM), BIX-01294 (1-100 μM), or a mixture of SAHA and BIX-01294 (1:1; 1-100 μM). At 10 μM concentrations when applied in combination (as a mixture) performance was enhanced towards MDA-MB-231 (EC₅₀ value of 1.891±0.56 versus 2.874±0.84 for SAHA alone or 2.155±0.88 for BIX-01294 alone) and was found to be comparable in MCF-7. Despite being distinct molecules with different physiochemical properties, application of both displayed a significant improvement (approximately 34% lower EC₅₀ to SAHA, and 13% lower EC₅₀ to BIX-01294 in MDA-MB 231). This provided the basis for exploring whether a single moiety capable of preserving the targeting activity of SAHA and BIX-01294 could be identified.

A multi-targeted therapy can be based on using two target-selective ligands as a base to provide a net therapeutic benefit greater than a single ligand. Two approaches can be pursued—either combining two active moieties as a cocktail or incorporating properly selected active moieties into a single molecule. Hybrid compounds, however, include a pharmacophore derived from two dissimilar compounds that can retain multiple functionalities inside the body. Hybrid drugs that target components belonging to the same scheme in disease progression or have otherwise interdependent functionality could yield an improved treatment effects.

As the lipophilic quinazoline core is similar to the lipophilic bulky cap for HDAC inhibitors, it was reasoned that the G9a core could function as the core scaffold of an HDAC and G9a dual inhibitor. Accordingly, the linker and the hydroxamic acid were added at the C₂, C₄, and C₇ position(s) of the quinazoline ring in order to obtain the desired hybrid molecules, as G9a has numerous inhibitors with bulky side chains, as in the case of E72. HDACIs can also afford a reasonable variety of lipophilic cores. Various analogs with different linker lengths and different groups at C₆ and at C₄ cyclohexylamine positions were also designed.

Considering the innate deficiency of HDACIs as a monotherapy it was hypothesized that the core metal ion binding hydrophilic segment could be coupled with the lipophilic core of G9a inhibitors in order to increase effectiveness. Both G9a and HDACs are therapeutic targets for cancer therapy, and are both capable of targeting identical substrates (H3K9 and lysine 373 of p53). In order to design compounds featuring both HDAC and G9a inhibition, the H3 mimicking quinazoline core of G9a inhibitors was used as a base scaffold with several modifications at several sites introduced to cover most of the possible chemical space with respect to the position and chain length (linker gap between metal binding portion and G9a core).

More than 25 compounds were tested biochemically and in vivo to determine for the desired dual inhibition activity. The primary assessment of success was made from MALDI-TOF evaluation of the H3K9 methylation profile; many of the compounds retained G9a inhibition potential. Cell-based assays of all the compounds against several cell lines were used to determine their inhibition potential. Compounds 13 and 14, in particular, were found to display the desired dual inhibition activities comparable to the controls SAHA and BIX-01294.

Cell toxicity of these compounds was determined using CCK-8, showing that compound 14 was both more effective and less toxic compared to 13.

### Example 2: Molecular Modelling of Dual Inhibitor Compounds

Methods:

Protein Preparation and Grid Generation:

The coordinates for the HDAC8/MS-344 complex (PDB ID: 1T67) and G9a/BIX-01294 complex (PDB ID: 3FPD) were downloaded from the RCSB Protein Data Bank. In these structures, MS-344 and BIX-01294 are bound to HDAC8, G9a respectively. The PDB protein-ligand structures were processed with the Protein Preparation Wizard in the Schrödinger suite. The protein structure integrity was checked and adjusted, and missing residues and loop segments near the active site were added using Prime. The receptor was prepared for docking by the addition of hydrogen atoms and the removal of co-crystallized molecules except for Zn²⁺, as it is near to the active site in the case HDAC. Active site water molecules outside 5.0 Å from the ligand were removed. The bound ligands were used to specify the active site. A 3D box was generated around each ligand to enclose the entire vicinity of active site. The receptor grid for each target was prepared with the help of OPLS_2005 force field. The grid center was set to be the centroid of the co-crystallized ligand, and the cubic grid had a size of 20 Å.

Ligand Preparation:

The 2D ligand structures were prepared using ChemBioDraw Ultra 12.0, and the 3D structures were generated by Schrödinger suite. Schrödinger's LigPrep program was used to generate different conformations of ligands. All possible protomers and ionization states were enumerated for 14 and bound ligands using Ionizer at a pH of 7.4. Tautomeric states were generated for chemical groups with possible prototropic tautomerism.

Molecular Docking:

Molecular docking studies were performed by using a GLIDE docking module of Schrödinger suite. It performs grid-based ligand docking with energetics and searches for positive interactions between ligand molecules and a typically larger receptor molecule, usually a protein. Finally, prepared ligands were docked into the generated receptor grids using Glide SP docking precision. The results were analyzed on the basis of the GLIDE docking score and molecular recognition interactions. All the 3-dimensional (3D) figures were obtained using Schrödinger Suite 2014-3.

Molecular Docking Analysis:

The assays showed that compound 14 had good cellular potency for inhibition of both G9a and HDAC, so docking was used to examine the interactions of compound 14 to the target proteins compared to known ligands using Schrödinger Suite 2014-3 (Friesner, J. Med. Chem. 2006, 49, 6177-6196). The crystal structure of human HDAC8 complexed with MS-344 (PDB ID: 1T67) and human G9a complexed with BIX-01294 (PDB ID: 3FPD) were selected as the templates for molecular docking studies (Chang et al., Nat. Struct. Mol. Biol. 2009, 16, 312-317; Somoza et al., Structure 2004, 12, 1325-1334). SP Glide algorithm was first validated by redocking MS-344 and BIX-01294 from the complex; ligand preparation was done using LigPrep with OPLS_2005. The search space was defined using Receptor Grid Generation in Glide, with the centroid of the complexed ligand chosen to define the grid box. Standard precision mode was selected for validation docking, and default settings for scaling van der Waals radii were used. No constraints were defined for the docking runs. The highest-scoring docking pose returned for MS-344 and BIX-01294 were compared with the starting protein complex. For subsequent molecular docking of compound 14 in the binding site of HDAC8 and G9a, LigPrep was used for energy minimizations of the molecule with the OPLS_2005 force field. Using the initial grids generated for HDAC8 and G9a, the standard precision docking was repeated for compound 14 as described above.

TABLE 5
GLIDE docking results for MS-344 and compound 14
at the catalytic site of HDAC8 (PDB ID: 1T67)

| | | | | Interactions | | |
|---|---|---|---|---|---|---|
| | | | | Hydrogen Bonds | | |
| S. | Ligand | Docking | GLIDE | Back- | Side | Interaction |
| No. | ID | Score | score | bone | Chain | with Zn²⁺ |
| 1 | MS- | −7.931 | −7.931 | His142, | Asp101, | Ionic |
| | 344 | | | His143, | Tyr306 | interaction |
| | | | | Gly151, | | |
| | | | | Gly304 | | |
| 2 | 14 | −7.934 | −8.369 | Gly140, | Asp101, | Ionic |
| | | | | His142, | Tyr306 | interaction |
| | | | | Gly151, | | |
| | | | | Gly304 | | |

TABLE 6
GLIDE docking results for BIX-01294 and compound
14 at the catalytic site of G9a (PDB ID: 3FPD)

| | | | | Interactions | | |
|---|---|---|---|---|---|---|
| | | | | Hydrogen Bonds | | |
| S. | Ligand | Docking | GLIDE | Back- | Side | Interaction |
| No. | Id | Score | Score | bone | Chain | with Zn²⁺ |
| 1 | BIX- | −7.664 | −8.134 | Ala1134 | Asp1131, | NA |
| | 01294 | | | | Asp1135, | |
| | | | | | Asp1140 | |
| 2 | 14 | −7.321 | −7.52 | Arg1137, | Asp1131, | NA |
| | | | | Glu1138 | Asp1135, | |
| | | | | | Asp1140, | |
| | | | | | Arg1214 | |

Tables 5 and 6 (above) show the results of docking along with principal interactions for compound 14 with HDAC8 and G9a. Predicted binding modes and the detailed protein-inhibitor interactions of compound 14 with HDAC8 and G9a were determined. The data showed that the catalytic tunnel of HDAC8 is occupied by the aliphatic side chain of the inhibitor, while the hydroxamate group chelates the zinc ion. The hydroxamate group also takes part in hydrogen-bonding interactions with residues in the catalytic tunnel. The zinc ion displays a trigonal bipyramidal geometry and with two points contact with the ligand. Docking studies suggest important structural/catalytic roles for Gly140, His142, Gly151 and Gly304 in the active site pocket and extending to Tyr306, Asp101. H-bond distances (A) between heteroatoms of ligand and amino acid residues are as follows: Asp101 (1.90), His142 (2.02), His143 (3.64), Gly151 (3.68), Gly304 (3.00), Tyr306 (2.17). Moreover, comparison of 14 and cocrystallized MS-344 suggests that 14 also occupies the binding pocket in a similar fashion to MS-344, effectively occupying the catalytic site of HDAC8.

A similar study was performed to establish the binding characteristics of compound 14 with G9a. The binding model of compound 14 indicated that it shares common hydrogen bonding interactions with key residues of the catalytic domain in a mode comparable to BIX-01294. Most notably, the piperidine ring substituted at quinazolin-4-amine in compound 14 has hydrogen bonding interactions with Arg1137, Glu1138 residues, and the aliphatic chain was involved in some more hydrogen bond interaction with the side chains of residues Asp1131, Asp1135, Asp1140 and Arg1214. H-bond distances (A) between heteroatoms of ligand and amino acid residues are as follows: Asp1131 (1.66), Asp1135 (1.75, 1.81), Arg1137 (3.33), Glu1138 (3.98), Asp1140 (1.77), Arg1214 (2.68, 2.90).

ADME Prediction Studies:

The procedures and principals from the in silico physico-chemical evaluations of known HDACIs were applied here to evaluate these novel dual inhibitors (Zang et al., J. Mol. Graph. Model. 2014, 54, 10-18). ADMET module of Discovery Studio 3.1 was used to predict physical properties. Using Lipinski's rule of five (Lipinski et al., Adv. Drug Deliv. Rev. 2001, 46, 3-26), the octanol-water partition coefficient (AlogP98) should be less than 5. As seen in Table 8, the candidate compound 14 is well within accordance of the rule. In addition, other values also fell into the acceptable ranges of PSA-2D (7-200) and QplogS (−6.5 to 0.5), indicating 14 may possess good bioavailability. These parameters were also taken into consideration in identifying better inhibitors, suggesting that 14 has the characteristics desirable in a drug candidate.

TABLE 7
ADME prediction results

| Entry | M.W | QPlogS^{c} | PSA | PSA-2D^{b} | AlogP98^{a} |
|---|---|---|---|---|---|
| 14 | 515.654 | −3.702 | 161.25 | 141.462 | 2.511 |
| SAHA | 264.324 | −2.139 | 102.256 | 81.037 | 1.838 |
| BIX- | 476.62 | −6.792 | 50.675 | 63.249 | 4.189 |
| 01294 | | | | | |

^{a}AlogP98 means atom-based LogP (octanol/water),
^{b}PSA-2D means 2D fast polar surface area.
^{c}QplogS means predicted aqueous solubility.

1T69 (HDAC) Protein Interaction Study:

The HDAC8 protein structure PDB ID: 1T69 was chosen for the modelling study because it has SAHA (which we used as the control in cell based assays) as the co-crystallized ligand, but the study revealed a lower GLIDE score and docking score than the expected, and so we did a similar study on another HDAC8 protein structure 1T67 and found a higher binding scores and chose this for later study.

TABLE 8
Glide docking study results for compound 14 and SAHA
at the catalytic site of HDAC 8 (PDB ID: 1T69)

| | | | | INTERACTIONS | | | |
|---|---|---|---|---|---|---|---|
| | LIGAND | GLIDE | DOCKING | H-bonds | | | Interaction |
| S. NO. | ID | SCORE | SCORE | Backbone | Side chain | π-π | with Zn²⁺ atom |
| 1 | SAHA | −5.794 | −5.794 | His142, His143, | Asp101, Tyr306 | Phe152 | + |
| | | | | Gly151, Gly304 | | | |
| 2 | 14 | −8.858 | −8.471 | Gly140, His42, | Asp101, Tyr306 | — | + |
| | | | | Gly151, Gly206, | | | |
| | | | | Phe207, Pro209, | | | |
| | | | | Gly304 | | | |

Unless defined otherwise, all technical and scientific terms used herein have the same meanings as commonly understood by one of skill in the art to which the disclosed materials and methods belongs. Publications cited herein and the materials for which they are cited are specifically incorporated by reference.

Those skilled in the art will recognize, or be able to ascertain using no more than routine experimentation, many equivalents to the specific forms of the materials and methods described herein. Such equivalents are intended to be encompassed by the following claims.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

We claim:

1. A compound of Formula I: 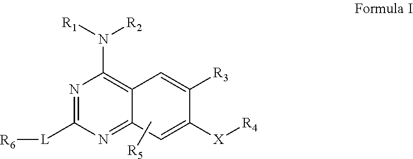
wherein X is absent or is oxygen (O), nitrogen (NH or NR₁₈) or sulfur (S);
wherein R₁ is hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or one of the moieties: 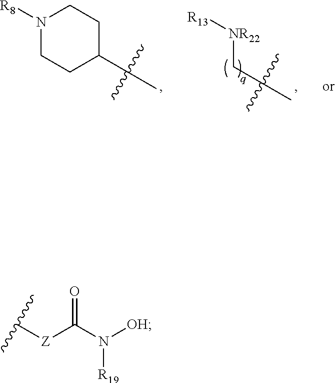
wherein q is an integer value in the range of 1-15, more preferably 1-10, most preferably 1-5;
wherein R₄, R₆, R₈, and R₁₃ are independently hydrogen, optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety: 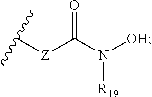
wherein Z is absent or a linking moiety, wherein the linking moiety is oxygen (O), nitrogen (NR₂₃), sulfur (S), optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;
wherein L is absent or a linking moiety, wherein the linking moiety is optionally substituted alkyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl;
wherein R₂, R₃, R₅, R₁₈, R₁₉, R₂₂, and R₂₃ are independently hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, or optionally substituted heteroaryl; and
wherein at least one of R₁, R₄, R₆, R₈, or R₁₃ is the moiety:

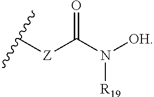

2. The compound of claim 1, wherein Z is: 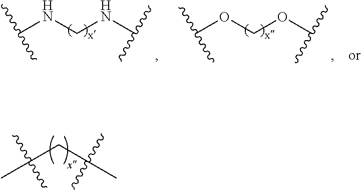
wherein x′, x″, and x′″ are integer values independently in the range of 1-15, more preferably 1-10, most preferably 1-5.

3. The compound of claim 1, wherein Z is absent and R₆ is: 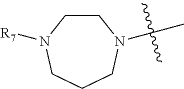
wherein R₇ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

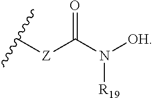

4. The compound of claim 1, wherein X is oxygen (O), and wherein R₁ is the moiety:

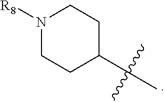

5. The compound of claim 4, wherein R₈ is a substituted or unsubstituted benzyl.

6. The compound of claim 4, wherein R₆ is: 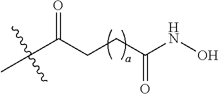
wherein a is an integer value in the range of 1-15, more preferably 1-10, most preferably 1-5.

7. The compound of claim 4, wherein R₆ is: 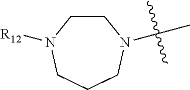
wherein R₁₂ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

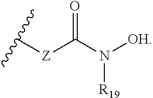

8. The compound of claim 4, wherein R₈ is: 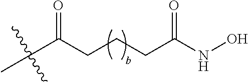
wherein b is an integer value in the range of 1-15, more preferably 1-10, most preferably 1-5.

9. The compound of claim 4, wherein R₄ is: 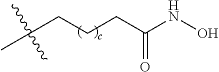
wherein c is an integer value in the range of 1-15, more preferably 1-10, most preferably 1-5.

10. The compound of claim 1, wherein X is oxygen (O), and wherein R₁ is the moiety:

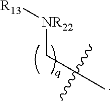

11. The compound of claim 10, wherein the R₆ is: 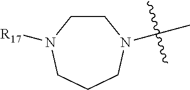
wherein R₁₇ is hydrogen, optionally substituted alkyl, optionally substituted alkoxyl, optionally substituted heteroalkyl, cycloalkyl, optionally substituted heterocycloalkyl, optionally substituted alkenyl, optionally substituted alkynyl, optionally substituted aryl, optionally substituted alkylaryl, optionally substituted heteroaryl, or the moiety:

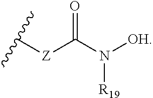

12. The compound of claim 10, wherein the R₁₃ is: 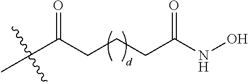
wherein d is an integer value in the range of 1-15, more preferably 1-10, most preferably 1-5.

13. The compound of claim 1, wherein the compound is: 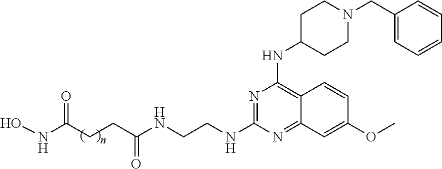
wherein n is an integer value in the range of 1-5.

14. The compound of claim 1, wherein the compound is:

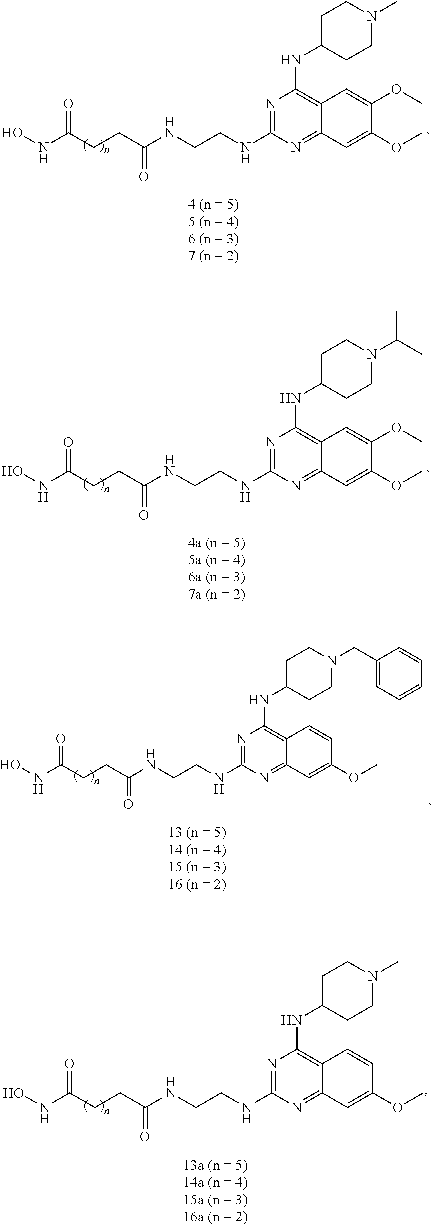

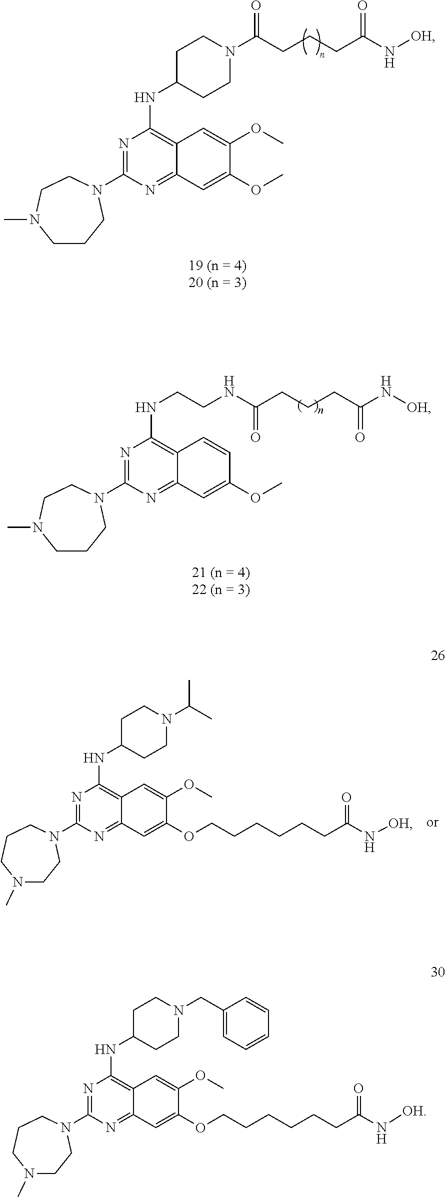

15. The compound of claim 1, wherein the compound inhibits both histone deacteylase and histone methyltransferase G9a.

16. A pharmaceutical composition comprising an effective amount of the compound of claim 1 in combination with a pharmaceutically acceptable diluent, excipient, or carrier.

17. A method of treating cancer in a subject in need thereof comprising administering an effective amount of the compound of claim 1.

18. The method of claim 17, wherein the compound is:

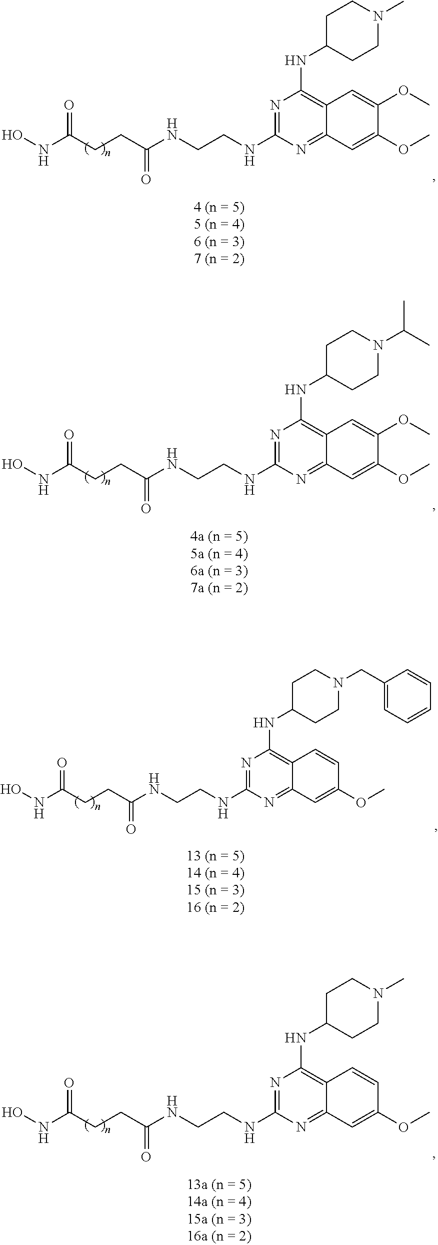

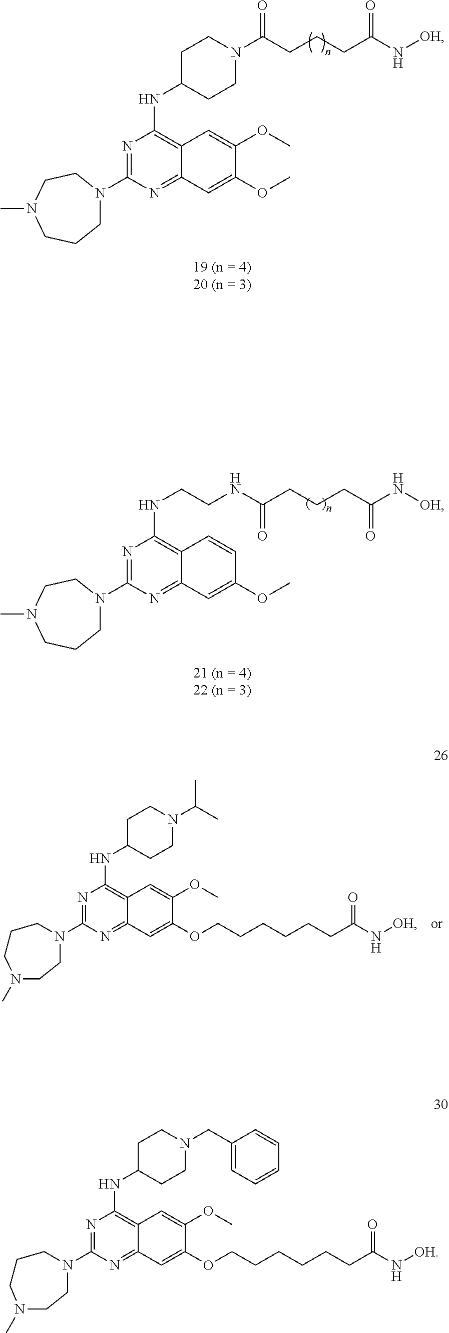

19. The method of claim 17, wherein the cancer is lung cancer, myeloma, leukemia, acute myeloid leukemia, carcinoma, hepatocellular carcinoma, lymphoma, breast cancer, prostate cancer, pancreatic cancer, cervical cancer, ovarian cancer, or liver cancer.
